(19) United States
(12) Patent Application Publication
(10) Pub. No.: US 2011/0097739 A1
(43) Pub. Date: Apr. 28, 2011

(54) BLOOD-BRAIN BARRIER EPITOPES AND USES THEREOF

(76) Inventors: Abedelnasser Abulrob, Ottawa (CA); Danica Stanimirovic, Greely (CA); Arumugam Muruganandam, Acton, MA (US)

(21) Appl. No.: 12/890,079

(22) Filed: Sep. 24, 2010

Related U.S. Application Data
(63) Continuation of application No. 12/088,337, filed on May 22, 2008.
(60) Provisional application No. 60/720,452, filed on Sep. 27, 2005.

(51) Int. Cl. G01N 33/53

(52) U.S. Cl. 435/7.8; 436/501

(57) ABSTRACT

The invention features a method of identifying an agent and generating an antibody that can cross the blood brain barrier, through the use of novel antigen isoforms of transmembrane domain protein 30A (TMEM30A). This is useful in establishing mechanisms of transmigration across the blood-brain barrier. These antigens are enriched in brain endothelium compared to other endothelial cells and may have better selectivity and capacity for brain delivery compared to transferrin and insulin receptors. One antigen is TMEM30A.

### PRIOR APPLICATION INFORMATION

This application claims the benefit of U.S. Provisional Application 60/720,452, filed Sep. 27, 2005.

### BACKGROUND OF THE INVENTION

Novel llama single-domain antibodies, FC5 and FC44, have been identified. These antibodies bind to antigens on the surface of brain endothelial cells and subsequently transmigrate into the brain. These antibodies and other binders having affinity for these epitopes are useful as ‘vectors’ to shuttle other molecules (therapeutics, diagnostics) into the brain.

Antibodies against receptors that undergo transcytosis across the blood-brain barrier have been used as vectors to target drugs or therapeutic peptides into the brain. A novel single domain antibody, FC5, has recently been identified which transmigrates across human cerebral endothelial cells in vitro and the blood-brain barrier in vivo. There is disclosed herein possible mechanisms of FC5 endocytosis and transcytosis across the blood brain barrier and its putative receptor on human brain endothelial cells as well as uses of FC5 and other such binders to this receptor. This receptor may be a new target for developing brain-targeting drug delivery vectors.

The brain capillary endothelium forms a formidable barrier to the entry of drugs into the central nervous system. The tight junctions that seal cerebral endothelial cells (CEC) prevent circulating compounds including therapeutic drugs from reaching the brain by the paracellular route. Other unique characteristics of CEC include lack of fenestrations, low number of pinocytic vesicles and an elaborate, highly negatively charged glycocalyx on their luminal surface. Further barrier to therapeutic brain delivery is the expression of efflux pumps and high enzymatic activity of CEC.

Biologics, including peptides, proteins and oligonucleotides could be delivered to the brain via vesicular transport across CEC known as transcytosis. This is a process that requires a specific or non-specific interaction of a ligand with moieties expressed at the luminal surface of CEC, which triggers internalization of the ligand into endocytic vesicles, their movement through the endothelial cytoplasm and exocytosis at the abluminal side of CEC. Different endocytic pathways have been described in CEC: a) macropinocytosis, a random pathway of internalization of large proteins, b) adsorptive-mediated endocytosis (AME) initiated through non-specific charge-based interactions of drugs/biologics with endothelial surface, and c) receptor-mediated endocytosis (RME) triggered by a specific interaction with receptors expressed on CEC. Both AME and RME have been exploited in designing drug-carrying vectors for delivery across the blood-brain barrier (BBB). For example, cationic cell-penetrating peptides, such as SynB vector family, have the ability to deliver hydrophilic molecules across the BBB via a temperature and energy-dependent AME process (Drin et al., 2003). Antibodies specific for brain endothelial antigens that undergo RME and transcytosis across the BBB, most notably anti-transferrin receptor antibody (OX26), have been used to shuttle biologics chemically linked to the antibody or encapsulated into antibody-functionalized carriers (e.g., immunoliposomes) across the BBB in experimental animal models.

There is currently a small number of known receptors expressed on brain endothelial cells that undergo receptor-mediated transcytosis: transferrin receptor, insulin receptor, low-density lipoprotein related protein receptor (LPR) and angiotensin II receptor. Of these, transferrin receptor and insulin receptor have been exploited to develop brain delivery vectors (i.e., antibodies that recognize these receptors). Although transferrin receptor is known to be enriched in brain endothelium compared to other organs, both transferrin and insulin receptors are widely distributed in other organs, and therefore, brain selectivity achieved by using these ‘targets’ is limited.

### SUMMARY OF THE INVENTION

According to a first aspect of the invention, there is provided a purified or isolated nucleic acid molecule comprising at least 75% identity to nucleotides of SEQ ID NO. 2.

According to a second aspect of the invention, there is provided a method of identifying an agent capable of TMEM30A-mediated transcytosis across the blood-brain barrier comprising:

incubating an agent of interest with a peptide comprising or having at least 75% identity to amino acids 1 to 361 of SEQ ID NO. 3 and detecting binding between said agent and said peptide; or

incubating an agent of interest with a peptide comprising or having at least 75% identity to amino acids 1 to 325 of SEQ ID NO. 4, and detecting binding between said agent and said peptide; or

incubating an agent of interest with a peptide comprising or having at least 75% identity to amino acids 1 to 242 of SEQ ID NO. 5 and detecting binding between said agent and said peptide; or

incubating an agent of interest with a peptide comprising or having at least 75% identity to amino acids 1 to 257 of SEQ ID NO. 6 and detecting binding between said agent and said peptide; or

incubating an agent of interest with a peptide comprising or having at least 75% identity to amino acids 1 to 40 of SEQ ID NO. 7 and detecting binding between said agent and said peptide; or

incubating an agent of interest with a peptide comprising or having at least 75% identity to amino acids 1 to 140 of SEQ ID NO. 8 and detecting binding between said agent and said peptide; or

incubating an agent of interest with a peptide comprising or having at least 75% identity to amino acids 1 to 18 of SEQ ID NO. 9 and detecting binding between said agent and said peptide; or

incubating an agent of interest with a peptide comprising or having at least 75% identity to amino acids 1 to 11 of SEQ ID NO. 10 and detecting binding between said agent and said peptide; or

incubating an agent of interest with a peptide comprising or having at least 75% identity to amino acids 1 to 11 of SEQ ID NO. 11 and detecting binding between said agent and said peptide; or

incubating an agent of interest with a peptide comprising or having at least 75% identity to amino acids 1 to 13 of SEQ ID NO. 12 and detecting binding between said agent and said peptide; or

incubating an agent of interest with a peptide comprising or having at least 75% identity to amino acids 1 to 13 of SEQ ID NO. 13 and detecting binding between said agent and said peptide; or

incubating an agent of interest with a peptide comprising or having at least 75% identity to amino acids 1 to 16 of SEQ ID NO. 14 and detecting binding between said agent and said peptide; or

incubating an agent of interest with a peptide comprising or having at least 75% identity to amino acids 1 to 16 of SEQ ID NO. 15 and detecting binding between said agent and said peptide.

According to a third aspect of the invention, there is provided a purified or isolated peptide comprising at least 75% identity to any one of the amino acid sequences as set forth in SEQ ID NO. 3, SEQ ID NO. 4, or SEQ ID NO. 5 or SEQ ID NO. 6 or SEQ ID NO. 7 or SEQ ID NO. 8 or SEQ ID NO. 9 or SEQ ID NO. 10 or SEQ ID NO. 11 or SEQ ID NO. 12 or SEQ ID NO. 13 or SEQ ID NO. 14 or SEQ ID NO. 15.

According to a fourth aspect of the invention, there is provided an isolated or purified peptide comprising 6 or more consecutive amino acids of any one of the amino acid sequences as set forth in SEQ ID NO. 3, SEQ ID NO. 4, or SEQ ID NO. 5. SEQ ID NO. 6 or SEQ ID NO. 7 or SEQ ID NO. 8 or SEQ ID NO. 9 or SEQ ID NO. 10 or SEQ ID NO. 11 or SEQ ID NO. 12 or SEQ ID NO. 13 or SEQ ID NO. 14 or SEQ ID NO. 15.

According to a fifth aspect of the invention, there is provided a method of generating an antibody capable of TMEM30A-mediated endocytosis and transcytosis across the blood-brain barrier comprising:

inoculating a subject with isolated or purified peptide comprising 6 or more consecutive amino acids of any one of the amino acid sequences as set forth in SEQ ID NO. 3, SEQ ID NO. 4, or SEQ ID NO. 5 or SEQ ID NO. 6 or SEQ ID NO. 7 or SEQ ID NO. 8 or SEQ ID NO. 9 or SEQ ID NO. 10 or SEQ ID NO. 11 or SEQ ID NO. 12 or SEQ ID NO. 13 or SEQ ID NO. 14 or SEQ ID NO. 15. and a suitable excipient such that an immune response against said peptide is generated; and recovering antibodies from said subject. Preferably, the subject is a non-human animal. As will be appreciated by one of skill in the art, means for generating an immune response against an antigen of interest using a variety of animals as subjects are known in the art. Specifically, immunization regimes, adjuvants, methods of antibody recovery, isolation and purification are all well known and well established for a large variety of subjects.

### BRIEF DESCRIPTION OF THE DRAWINGS

FIG. 1. Accumulation of FC5 antibody in the brain after i.v. injection into mice determined by optical imaging.

(A) FC5 or NC11 were conjugated to Cy5.5 near infrared probe and then injected (3 nM) by tail vein into the animal for 6 hours. Head imaging showed higher accumulation of FC5 compared to NC11 or the fluorophores alone. (B) Quantification of the head region of interest average fluorescence concentration after injection of FC5 or NC11 or Cy5.5 alone. (C) Dorsal body imaging of the whole animal after injection of FC5 or NC11 or Cy5.5 alone. (D) Quantification of the organs region of interest average fluorescence concentration after injection of FC5 or NC11 or Cy5.5 alone. (E) Ex-vivo brain imaging of FC5 or NC11 or Cy5.5 injected animals after kill perfusion demonstrates the higher accumulation of FC5 antibody in the brain.

FIG. 2. Describes conjugation of the blood-brain barrier permeable sdAb FC5 with mouse IgG tagged with horse-radish peroxidase (IgG-HRP) and functional evaluation of the construct in vitro. Additional cysteine moiety was added to FC5 by genetic engineering as described in Materials and Methods. A) cysFC5 was conjugated with maleimide-activated IgG-HRP as in shown reaction. B&C) Uptake of IgG-HRP (B) or FC5-IgG-HRP conjugate (C) in human brain endothelial cells in culture. Cells were fixed 30 min after addition of 5 μg/ml of either conjugate. Uptake was determined in fixed cells using an FITC-labelled anti-mouse secondary antibody Materials and Methods. D) Transmigration of IgG-HRP (▴) or FC5-IgG-HRP conjugate (▪) across the in vitro blood-brain barrier model. Transport studies were performed as described in Materials and Methods.

FIG. 3. A) Polarized transmigration of FC5 across in vitro blood-brain barrier (BBB) model. Transport studies were initiated by adding 10 μg/ml FC5 to either apical (A→B) or basolateral (B→A) compartment and the amount of FC5 in the opposite compartment was determined after 30 minutes as described in Materials and Methods. ¹⁴C-sucrose distribution across the same HCEC monalayers was used as internal control for paracellular transport. B) Effects of pharmacological inhibitors of adsorptive-mediated endocytosis (AME) and macropinocytosis on transmigration of FC5 across in vitro BBB model. HCEC were pretreated for 30 minutes with AME inhibitors, protamine sulfate (40 μg/ml) and poly-1-lysine (300 μM), or micropinocytosis inhibitor, amiloride (500 μM), and FC5 transport was measured over 30 minutes as described in Materials and Methods. Each bar represents mean±s.d. from 6 replicate membranes.

FIG. 4. Energy-dependence of FC5 uptake into HCEC and transmigration across in vitro blood-brain barrier model. Confocal microscopy images of FC5 uptake into HCEC at 37° C. (A) and at 4° C. (B). Cells were exposed to 5 μg/ml FC5 for 30 minutes and processed for double immunochemistry for c-myc tag of FC5 as described in Materials and Methods. C) Transcellular migration of 10 μg/ml FC5 across HCEC at 37° C. or 4° C., or after a 30-min exposure of HCEC to 5 mM NaN₃ and 5 mM deoxyglucose (2DG) for 20 min in glucose-free medium. FC5 transmigration was determined 30 min after addition to HCEC as described in Materials and Methods. D) The effect of Na⁺,K⁺-ATPase inhibitor, ouabain, on transcellular migration of FC5 across HCEC. Cells were pre-treated with 1 μM ouabain for 30 minutes and FC5 transport was measured over 30 minutes as described in Materials and Methods. Each bar represents mean±s.d. from 6 replicate membranes. Asterisks indicate significant differences (P<0.05; Student's t-test) from 37° C. or untreated cells.

FIG. 5. Role of clathrin-coated pits and caveolae in endocytosis and transcytosis of FC5 in HCEC. Colocalization of FC5 (green fluorescence) (A) and clathrin (red fluorescence) (B) in HCEC cells. Overlay image is shown in (C). Colocalization of FC5 (green fluorescence) (D) and caveolin-1 (red fluorescence) (E). Overlay image is shown in (F). Cells were exposed to FC5 for 30 minutes, washed and processed for double immunocytochemistry as described in Materials and Methods. Images are representative of 3-5 separate experiments. G) Western blots showing distribution of caveolin-1, FC5, and clathrin heavy chain immunoreactivity in subcellular fractions obtained from HCEC exposed to FC5 for 30 minutes. HCEC cells were fractionated as described in Materials and Methods. Western blots are representative of 3 separate experiments. H) Effects of pharmacological inhibitors of caveolae-mediated endocytosis, methyl-β-cyclodextrin (5 mM), nystatin (5 μg/ml) and filipin (5 μg/ml), or inhibitors of clathrin-coated pits-mediated endocytosis, chlorpromazine (50 μg/ml) or potassium-free buffer on transmigration of FC5 across in vitro BBB model. Human CEC were pretreated for 30 minutes with above inhibitors and FC5 transport was measured over 30 minutes as described in Materials and Methods. Each bar represents mean±s.d. from 6 replicate membranes. Asterisks indicate significant differences (P<0.05; one-way ANOVA, followed by Dunnett's multiple comparison between means).

FIG. 6. FC5 processing in endosomes. Colocalization of FC5 (green fluorescence) (A) and Texas red-conjugated tranferrin (red fluorescence) (B) in HCEC cells. Overlay image is shown in (C). Colocalization of internalized FC5 (green fluorescence) (D) and cathepsin-B (red fluorescence) (E) in HCEC cells. Overlay image is shown in (F). CEC are processed for immunochemistry and confocal microscopy as described in Materials and Methods. G) Western blot of FC5 prior to (top) and after (bottom) transcytosis across HCEC in vitro BBB model. H) Transcellular migration of 10 μg/ml FC5 across HCEC pre-treated with 25 μM monensin for 30 minutes. Transport studies were performed as described in Materials and Methods.

FIG. 7. A) Role of cytoskeletal network in FC5 transcytosis across HCEC. HCEC were pretreated for 30 minutes with the actin microfilament inhibitors cytochalasin D (0.5 μM) or latrunculin A (0.1 μM) or with the microtubule inhibitors nocodazole (20 μM) or colchicine (20 μM) and FC5 tranmigration across in vitro BBB model was measured over 30 minutes as described in Materials and Methods. B) Signaling pathway modulators wortmannin (0.5 μM), BIM-1 (5 μM), genistein (50 μM) or dbcAMP (500 mM) were added to HCEC 30 minutes before addition of 10 μg/ml FC5, and transcytosis across in vitro BBB model was measured after 30 minutes. Each bar represents mean±s.d. from 6 replicate membranes. Asterisks indicate significant differences (P<0.05; one-way ANOVA, followed by Dunnett's multiple comparison between means) from control.

FIG. 8. Role of oligossacharide antigenic epitopes in FC5 uptake into and transcytosis across HCEC. A-D) Fluorescent micrographs of FC5 uptake in HCEC in the absence (A) or presence of 100 μg/ml WGA (B), 200 μM sialic acid (C) or 0.1 U neuraminidase (D). Uptake was measured over 30 minutes. E) Transcytosis of 10 μg/ml FC5 across HCEC pre-treated with 200 μM sialic acid or indicated concentrations of neuraminidase for 30 minutes. F) Transccytosis of 10 μg/ml FC5 across HCEC pre-treated with 100 μg/ml WGA, 100 μg/ml Sambucus nigra agglutinin (SNA) or 100 μg/ml Maackia amurensis agglutinin (MAA) for 30 minutes. Transport studies were performed as described in Materials and Methods. G) FC5 binding to isolated protein (black bars) and lipid (gray bars) fractions of HCEC determined by ELISA. Prior to fractionation, lysed cells were incubated in the absence or presence of 1 U/ml neuraminidase for 1 hour at 37° C. ELISA on isolated protein and lipid fractions was performed as described in Materials and Methods. Each bar represents mean±s.d. from 6 replicates. Asterisks indicate significant differences (P<0.05; one-way ANOVA, followed by Dunnett's multiple comparison between means) from control.

FIG. 9. Lack of transferrin receptor involvement in FC5 transcytosis across in vitro BBB. A) Binding of the anti-transferrin receptor monoclonal antibody, CD71, FC5, pentameric construct of FC5 (P5) or non-related antibody from the same library that recognizes carbohydrate antigen, CEA, to human tranferrin receptor immobilized onto ELISA plate. The plates were read at 450 nm with an automated microplate reader. B) Western blot of human transferrin receptor immunodetected by anti-CD71, but not by P5. C) Transcytosis of 10 □g/ml FC5 alone or in the presence of 100-fold (1 mg/ml) of holotransferrin across HCEC monolayers. Transport was measured over 30 minutes as described in Materials and Methods. Each bar represents mean±s.d. from 6 replicate determinations.

FIG. 10. A combination of genomics and proteomics strategies used in FC5 antigen identification.

FIG. 11. Tissue distribution of the putative FC5 antigen

FIG. 12. TMEM 30A gene expression in various cell types

FIG. 13. Expression of TMEM30A in HEK293 cells.

FIG. 14. Recognition of TMEM30A by FC5 in cell lysate of TMEM30A overexpressing cells

FIG. 15. Competition of TMEM30A-mediated transmembrane transport of phosphatidyl-choline in human brain endothelial cells by FC5.

### DESCRIPTION OF THE PREFERRED EMBODIMENTS

Using a combination of cell biology, biochemistry, immunochemistry and molecular biology techniques, novel antigens related to the blood-brain barrier have been identified. This is useful in establishing mechanisms of transmigration across the blood-brain barrier. These antigens are enriched in brain endothelium compared to other endothelial cells and may have better selectivity and capacity for brain delivery compared to transferrin and insulin receptors.

In the examples, single domain antibody FC5, recognizing blood-brain barrier antigen and undergoing transmigration across the blood brain barrier is discussed.

While the invention is not limited to any particular mechanism or mode of action, the postulated mechanism is set out below for general interest.

### Mechanism of FC5 Transmigration Across the BBB:

1. Upon binding to its putative receptor on brain endothelial cells, FC5 transmigrates across by a mechanism known as receptor-mediated transcytosis.
2. FC5 is internalized into and transmigrates across brain endothelium in clathrin-coated pits.
3. Transmigration of FC5 is energy-dependent and saturable
4. Intact cytoskeleton network is necessary for FC5 transmigration
5. Transmigration of FC5 is dependent on PI3 kinase activity

Also described is the isolation and identification of the FC5 antigen, TMEM30A (SEQ ID NO: 2). As discussed herein, binding of the FC5 antigen to TMEM30A results in transmigration of the FC5 antibody across the blood-brain barrier.

### Antigen Recognized by FC5:

1. α(2,3)-linked sialic acid residues are a component of the antigenic epitope recognized by FC5
2. Antigen recognized by FC5 is sialiated protein and not sialiated lipid (ganglioside)
3. Recognition of α(2,3)-linked sialic acid residues on the putative protein antigen by FC5 is necessary for FC5 endocytosis and transmigration across brain endothelial cells
4. α(2,3)-linked sialic acid residues are only a component of the full antigen recognized by FC5
5. Transferrin receptor is not recognized by FC5
6. SEQ ID NO: 1 pulled out by panning of phage-displayed human brain cDNA expression library against FC5.
7. Gene blast the SEQ ID NO.2 aligned with TMEM30A (NM_{—}018247).
8. Tissue distribution of FC5 antigen is shown in FIG. 11. Strong expression was observed in brain tissues.
9. Cell distribution of TMEM30A mRNA is shown in FIG. 12. Strong expression is shown in endothelial cells.
10. TMEM30A over-expressed in HEK293 cells was immunoprecipited by FC5 pentamer (FIG. 14).

Thus it has been demonstrated that compounds or molecules or agents that bind to TMEM30A are capable of TMEM30A-mediated translocation across the blood-brain barrier. Consequently, in one embodiment, there is provided a method of identifying agents capable of crossing the blood-brain barrier comprising providing an agent of interest and determining if said agent binds to TMEM30A as described below.

In yet other embodiments, there is provided a method of identifying agents capable of TMEM30A translocation across the blood-brain membrane comprising exposing TMEM30A peptide as described below to an agent of interest under conditions suitable for binding of the agent to the TMEM30A peptide and then determining if binding has occurred. As discussed herein, binding or interaction may be determined by a variety of means, for example, by retention of the agent on a column or other similar support having TMEM30A as described below mounted thereto, or by demonstrating translocation using the in vitro cell assay or in vivo assay described herein. It is of note that these assays are for illustrative purposes and one skilled in the art will understand that there are a wide variety of ways to detect interaction between an agent of interest and TMEM30A.

In yet other embodiments, there is provided a method of identifying agents capable of interaction with TMEM30A comprising exposing TMEM30A peptide as described below to an agent of interest under conditions suitable for binding of the agent to the TMEM30A peptide and then determining if binding has occurred. As will be appreciated by one of skill in the art, such an agent may be used for a variety of purposes, for example, membrane transport, imaging and the like, as discussed herein.

As will be appreciated by one skilled in the art, determination of binding to TMEM30A may be done several ways. For example, a high through-put initial screen may be done wherein for example a column is loaded with TMEM30A and agents of interest are passed through the column. Retained compounds could then be eluted and investigated further, for example, in the in vitro or in vivo assays described below.

It is of note that such agents can be combined, joined, crosslinked or otherwise attached to a compound of interest, thereby forming a conjugate which can be translocated across the blood-brain barrier.

In some embodiments, the compound of interest may be a detectable compound for example but by no means limited to a radiolabel, an isotope, a visible or near-infrared fluorescent label, a reporter molecule, biotin or the like. As will be appreciated by one skilled in the art, such conjugates may be used for confirmation that the agent is translocating or for imaging or for other similar purposes.

In other embodiments, the compound of interest is a small molecule, for example, an anti-cancer drug, for example but by no means limited to paclitaxel, vinblastine, vincristine, etoposide, doxorubicin, cyclophosphamide, chlorambucil or the like.

In yet other embodiments, the small molecule may be a therapeutic or pharmaceutical compound for treating a neurological disease, for example, a brain tumor, a brain metastasis, schizophrenia, epilepsy, Alzheimer's disease, Parkinson's disease, Huntington's disease, stroke, obesity, multiple sclerosis and the like.

As discussed herein, FC5 antibody binds to TMEM30A. As such, peptides comprising 6 or more, 7 or more, 8 or more, 9 or more or 10 or more consecutive amino acids of SEQ ID NO: 3 may be used to generate monoclonal antibodies which recognize FC5. In some embodiments, the peptides are preferentially from the extracellular domain of TMEM30A, that is, from amino acids 67-323 of SEQ ID NO. 2. Similarly, the extracellular domain of isoform 2 (SEQ ID No. 4) corresponds to amino acids 67-287 of SEQ ID No. 4 and isoform 3 (SEQ ID No. 5) has an extracellular domain from amino acids 1-204 of SEQ ID No. 5. According, in other preferred embodiments, the peptides correspond to regions of these extracellular domains from isoforms 2 and 3. Thus, in some embodiments, the agent of interest may be a monoclonal antibody directed against an immunogenic fragment of TMEM30A as described herein. It is of note that other suitable fragments will be readily apparent to one skilled in the art. For example, a peptide comprising 6 or more, 7 or more, or more, 9 or more, or 10 or more consecutive amino acids from regions of the TMEM30A comprising the glycosylation sites, for example, as set forth in SEQ ID No. 7 or SEQ ID No. 8, may be used in some embodiments. Alternatively, regions highly conserved between TMEM30A and other evolutionarily similar peptides may also be used preferentially as discussed above, for example, as set forth in SEQ ID Nos 9-15.

In yet other embodiments, there is provided a purified or isolated nucleotide sequence having at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to nucleotides as set forth in SEQ ID NO: 1.

In yet other embodiments, there is provided a purified or isolated nucleotide sequence having at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to nucleotides 141 to 1226 as set forth in SEQ ID NO: 2. As will be appreciated by one of skill in the art, such nucleotide sequences may be used in expression systems for preparation of TMEM30A peptides as discussed herein or may be used as probes, primers or the like as discussed herein.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-361 or 1-323 or 67-323 as set forth in SEQ ID NO: 3.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-325 or 67-287 as set forth in SEQ ID NO: 4.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-242 or 1-204 as set forth in SEQ ID NO: 5.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-257 as set forth in SEQ ID NO: 6.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-40 as set forth in SEQ ID NO: 7.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-140 as set forth in SEQ ID NO: 8.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-18 as set forth in SEQ ID NO: 9.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-11 as set forth in SEQ ID NO: 10.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-11 as set forth in SEQ ID NO: 11.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-13 as set forth in SEQ ID NO: 12.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-13 as set forth in SEQ ID NO: 13.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-16 as set forth in SEQ ID NO: 14.

In yet other embodiments, there is provided a purified or isolated peptide comprising or having an amino acid sequence that is at least 75% identical or at least 76% or at least 77% or at least 78% or at least 79% or at least 80% or at least 81% or at least 82% or at least 83% or at least 84% or at least 85% or at least 86% or at least 87% or at least 88% or at least 89% or at least 90% or at least 91% or at least 92% or at least 93% or at least 94% or at least 95% or at least 96% or at least 97% or at least 98% or at least 99% identical to amino acids 1-16 as set forth in SEQ ID NO: 15.

As discussed herein, TMEM30A isoform 1, SEQ ID No. 3, has an internal C-terminus (amino acids 1-42), a transmembrane domain (amino acids 43-66) and an external domain (amino acids 67-323). As will be appreciated by one of skill in the art, modifications within the transmembrane domain must conserve the membrane spanning function or this peptide will likely be defective. Similarly, additions, deletions and substitutions within the C-terminus are more likely to be tolerated than at the extracellular N-terminus. It is noted that as discussed herein there exist at least two splicing variants of TMEM30A which strongly suggests that large variations for example insertions and deletions may be tolerated.

TMEM30A isoform 2, SEQ ID No. 4, has two transmembrane regions: amino acids 44-66 and amino acids 288-310; and amino acids 67-287 are external.

TMEM30A isoform 3, SEQ ID No. 5, has one transmembrane region at amino acids 205-227 of SEQ ID No. 5 and an external domain of amino acids 1-204 of SEQ ID No. 5.

In yet other embodiments, there is provided a nucleic acid molecule comprising a nucleotide sequence deduced from any one of the above peptides or amino acid sequences. These nucleic acid molecules may be used as discussed above, for example, for expression, as probes or primers or the like.

In addition to the full-length sequence TMEM30A polypeptides described herein, it is contemplated that TMEM30A variants can be prepared. TMEM30A variants can be prepared by introducing appropriate nucleotide changes into the TMEM30A DNA, and/or by synthesis of the desired TMEM30A polypeptide. Those skilled in the art will appreciate that amino acid changes may alter post-translational processes of the TMEM30A, such as changing the number or position of glycosylation sites or altering the membrane anchoring characteristics.

In addition the TMEM30A variant can have one or more other modifications, such as an amino acid substitution, an insertion of at least one amino acid, a deletion of at least one amino acid, or a chemical modification. For example, the invention provides a TMEM30A variant that is a fragment. In a variation of this embodiment, the fragment includes residues corresponding to a portion of human TMEM30A extending from about residue 67 to about residue 323 of SEQ ID No. 3.

Variations in the full-length sequence TMEM30A or in various domains of the TMEM30A described herein, can be made, for example, using any of the techniques and guidelines for conservative and non-conservative mutations. Variations may be a substitution, deletion or insertion of one or more codons encoding the TMEM30A that results in a change in the amino acid sequence of the TMEM30A as compared with the native sequence TMEM30A. Optionally the variation is by substitution of at least one amino acid with any other amino acid in one or more of the domains of the TMEM30A. Amino acid substitutions can be the result of replacing one amino acid with another amino acid having similar structural and/or chemical properties, such as the replacement of a leucine with a serine. Insertions or deletions may optionally be in the range of about 1 to 5 amino acids.

### TMEM30A Anti-Sense Oligonucleotides

Any TMEM30A sequences disclosed in the present application may similarly be employed as probes. Fragments of the TMEM30A nucleic acids can be useful to design antisense or sense oligonucleotides comprising a singe-stranded nucleic acid sequence (either RNA or DNA) capable of binding to target TMEM30A mRNA (sense) or TMEM30A DNA (antisense) sequences. Antisense or sense oligonucleotides comprise a fragment of the coding region of TMEM30A DNA as described above. Such a fragment generally comprises at least about 14 nucleotides, preferably from about 14 to 30 nucleotides. The ability to derive an antisense or a sense oligonucleotide, based upon a cDNA sequence encoding a given protein is described in, for example, (Cohen J S. Oligonucleotide therapeutics. Trends Biotechnol. 1992 March; 10(3):87-91.). Binding of antisense or sense oligonucleotides to target TMEM30A nucleic acid sequences results in the formation of duplexes that block transcription or translation of the TMEM30A sequence by one of several means, including enhanced degradation of the duplexes, premature termination of transcription or translation, or by other means. The antisense oligonucleotides thus may be used to block expression of TMEM30A protein which will modulate brain drug delivery. TMEM30A antisense or sense oligonucleotides further comprise oligonucleotides having modified sugar-phosphodiester backbones and wherein such sugar linkages are resistant to endogenous nucleases and therefore more suitable for in vivo applications.

### Uses for Anti-TMEM30A Antibodies

The following examples are offered for illustrative purposes only, and are not intended to limit the scope of the present invention in any way.

The anti-TMEM30A antibodies of the invention have various utilities. For example, anti-TMEM30A antibodies may be used in diagnostic assays for TMEM30A, e.g., detecting its expression (and in some cases, differential expression) in specific cells, tissues, or serum. Various diagnostic assay techniques known in the art may be used, such as competitive binding assays, direct or indirect sandwich assays and immunoprecipitation assays. The antibodies used in the diagnostic assays can be labeled with a detectable moiety. The detectable moiety may be a radioisotope ³²P, a fluorescent or chemiluminescent compound such as rhodamine or luciferin, or an enzyme, such as alkaline phosphatase, or horseradish peroxidase. Methods for conjugating the antibody to the detectable label are known in the art.

Anti-TMEM30A antibodies also are useful for the affinity purification of TMEM30A from recombinant cell culture or natural sources. In this process, the antibodies against TMEM30A are immobilized on a suitable support, such a Sephadex resin, using methods well known in the art. The immobilized TMEM30A antibody then is contacted with a sample containing the TMEM30A to be purified, and thereafter the support is washed with a suitable solvent that will remove substantially all the material in the sample except the TMEM30A, which is bound to the immobilized antibody. Finally, the support is washed with another suitable solvent that will elute the purified TMEM30A.

### Bi-Functional Antibodies

Bispecific antibodies (monoclonal, single chain, single domain or other fragments), preferably human or humanized, antibodies that have binding specificities for at least two different antigens. In the present case, one of the binding specificities is for TMEM30A, the other one is for any other brain antigen, and preferably for a neuronal cell-surface protein or neuronal receptor or neuronal receptor subunit.

### Use of TMEM30A for Drug Screening

This invention is particularly useful for screening compounds by using TMEM30A polypeptides or fragment thereof in any of a variety of drug screening techniques. The TMEM30A polypeptide or fragment employed in such a test may either be free in solution, affixed to a solid support, or borne on a cell surface. One method of drug screening utilizes eukaryotic or prokaryotic host cells which are stably transformed with recombinant nucleic acids expressing the TMEM30A polypeptide or fragment. Drugs are screened against such transformed cells in competitive binding assays. Such cells, either in viable or fixed form, can be used for standard binding assays. One may measure, for example, the formation of complexes between TMEM30A polypeptide or a fragment and the agent being tested, or one can examine the enhancement of internalization of the agent being tested following binding to TMEM30A polypeptide or a fragment. Alternatively, one can examine the diminution in internalization of TMEM30A polypeptide in its target cell caused by the agent being tested.

Thus, the present invention provides methods of screening for drugs or any other agents which can affect TMEM30A polypeptide or a fragment of it resulting in enhancement of the internalization of the tested drug in cells. These methods comprise contacting such an agent with TMEM30A polypeptide or fragment thereof and assaying for the presence of a complex between the agent and the TMEM30A polypeptide or fragment, or for the presence of a complex between the agent and TMEM30A polypeptide or fragment intracellularly, by methods well known in the art. In such competitive binding assays, the agent or TMEM30A polypeptide or fragment is typically labeled. After suitable incubation, free TMEM30A polypeptide or fragment is separated from that present in bound form, and the amount of free or uncomplexed label is a measure of the ability of the particular agent to bind to TMEM30A polypeptide.

The present invention also provides methods of screening for drugs or any other agents which can affect TMEM30A polypeptide expression or function resulting in cerebrovascular associated diseases. These methods comprise contacting such an agent with TMEM30A polypeptide or fragment thereof and assaying for the presence of a complex between the agent and the TMEM30A polypeptide or fragment, or for the presence of a complex between the agent and TMEM30A polypeptide or fragment intracellularly, by methods well known in the art. In such competitive binding assays, the agent or TMEM30A polypeptide or fragment is typically labeled. After suitable incubation, free TMEM30A polypeptide or fragment is separated from that present in bound form, and the amount of free or uncomplexed label is a measure of the ability of the particular agent to bind to TMEM30A polypeptide.

Another technique for drug screening provides high throughput screening for compounds having suitable binding affinity to TMEM30A polypeptide. For example, different small peptide test compounds are synthesized on a solid substrate. As applied to a TMEM30A polypeptide, the peptide test compounds are reacted with TMEM30A polypeptide and washed. Bound TMEM30A polypeptide is detected by methods well known in the art. Purified TMEM30A polypeptide can also be coated directly onto plates for use in drug screening techniques. In addition, TMEM30A non-neutralizing antibodies such as FC5 can be used to capture the TMEM30A polypeptides or fragments and immobilize it on the solid support.

This invention also contemplates the use of competitive drug screening assays in which neutralizing antibodies capable of binding TMEM30A polypeptide specifically (example FC5) compete with a test compound for binding to TMEM30A polypeptide or fragments thereof. In this manner, the antibodies can be used to detect the presence of any peptide which shares one or more antigenic determinants with TMEM30A polypeptide

Rational Drug Design: The goal of rational drug design is to produce structural analogs of biologically active TMEM30A or of small molecules with which they interact with TMEM30A, e.g., agonists, antagonists, or inhibitors. Any of these examples can be used to fashion drugs which are more active or stable forms of the TMEM30A polypeptide or which enhance brain drug delivery in vivo.

In one approach, the three-dimensional structure of the TMEM30A polypeptide, or of TMEM30A polypeptide-agent complex, is determined by x-ray crystallography, or by computer modeling. Less often, useful information regarding the structure of the TMEM30A polypeptide may be gained by modeling based on the structure of homologous proteins such as TMEM30B [GeneBank NM_{—}001017970]. In both cases, relevant structural information is used to design analogous TMEM30A polypeptide-like molecules or to identify efficient modulators that have improved stability or activity to improve drug delivery.

### Identification of TMEM30A/Ligand Interactions

Agents can be tested for their ability to bind to TMEM30A polypeptide or fragments for the purpose of identifying receptor/ligand interactions. The identification of a ligand for TMEM30A would be useful for a variety of indications including, for example, targeting bioactive molecules (linked to the ligand or TMEM30A) to a cell known to express the receptor such as brain endothelial cells for the purpose of brain drug delivery, use of TMEM30A or ligand as a reagent to detect the presence of the ligand or TMEM30A in a composition suspected of containing the same, wherein the composition may comprise cells suspected of expressing the ligand or TMEM30A, modulating the biological activity of a cell known to express or respond to the TMEM30A or ligand, modulating the permeability of cells that express TMEM30A to drugs, or allowing the preparation of agonists, antagonists and/or antibodies directed against TMEM30A or ligand which will modulate the permeability, or other biological activity of a cell expressing TMEM30A, and various other indications which will be readily apparent to the ordinarily skilled art. For example an epitope-tagged potential ligand such as poly-histidine tag is allowed to interact with TMEM30A. Following a 1 hour co-incubation with the epitope tagged peptide agent, TMEM30A is immunoprecipitated with protein A beads and the beads are washed. Potential ligand interaction is determined by western blotting of the complex with antibody directed towards the epitope tag.

Thus, in an embodiment of the invention there is provided a method of causing or enhancing movement of a cargo substance across the blood-brain barrier, said method comprising:
a) obtaining a binder having affinity for a blood-brain barrier antigen;
b) functionally linking the cargo substance to the binder (for example by conjugation or by encapsulating the cargo molecule in a liposome or other suitable capsule having a binder on its surface;
c) allowing contact between the binder and brain endothelial cells.

It will be understood that a cargo substance may be any compound of interest, including a pharmaceutical, an imaging agent, a toxin, or another suitable compound.

In some instances it may be desirable to include one or more molecules having affinity for a target accessible after transmigration of the blood brain barrier, to facilitate specific targeting of the cargo substance.

Receptors that undergo receptor-mediated transcytosis across the blood-brain barrier (such as antigen recognized by FC5) can be utilized to deliver drugs/therapeutics into the brain by developing various ligands that cluster the receptors and stimulate their transmigration. These are typically antibodies, but could be peptides, oligosaccharides, etc.

### EXAMPLES

To discover new antigen-ligand systems that can be exploited for transvascular brain delivery, a llama single-domain antibody (sdAb) phage-display library (Tanha et al., 2002) was used for differential antigen selection between human lung and brain microvascular endothelial cells. sdAbs are V_{H}H fragments of the heavy chain IgGs, which occur naturally and lack light chain, and are half the size (13 kDa) of a single-chain antibody (scFv). Two novel sdAbs, FC5 (GenBank No. AF441486) and FC44 (GenBank No. AF441487), which selectively recognized HCEC and transmigrated across the BBB in vitro and in vivo, were isolated in these studies. These sdAbs were engineered to enable their conjugation with biologics and carriers (Abulrob et al, 2005). sdAbs have several advantages over conventional antibodies as potential transvascular brain delivery vectors including their small size, low non-specific interactions with tissues expressing high levels of Fc receptors (e.g., liver, spleen) and thus low immunogenicity, and remarkable stability against high temperature, pH, and salts.

### Example 1

### FC5 ‘Targets’ the Brain After Intravenous Injection In Vivo

To investigate biodistribution of FC5, FC5 was conjugated with the near-infrared probe, Cy5.5, through NHS ester linkage and injected in mice intravenously via the tail vein. Mice were imaged by small animal time-domain eXplore Optix pre-clinical imager

(GE Healthcare). Animals were either injected with the near-infrared fluorescent probe, Cy5.5 alone or conjugated to FC5 (50 μg) or negative control antibody NC11 (50 μg) via tail vein using a 0.5-ml insulin syringe with a 27-gauge fixed needle. Animals were then imaged in eXplore Optix 6 h after drug injection. In all imaging experiments, a 670-nm pulsed laser diode with a repetition frequency of 80 MHz and a time resolution of 250 ps light pulse was used for excitation. The fluorescence emission at 700 nm was collected by a highly sensitive time-correlated single photon counting system and detected through a fast photomultiplier tube offset by 3 mm for diffuse optical topography reconstruction. Each animal was positioned prone on a plate that was then placed on a heated base (36° C.) in the imaging system. A two-dimensional mid-body scanning region encompassing the head was selected via a top-reviewing real-time digital camera. The optimal elevation of the animal was verified via a side viewing digital camera. The animal was then automatically moved into the imaging chamber for laser scanning. Laser excitation beam controlled by galvomirrors was then moved over the selected ROI. Laser power and counting time per pixel were optimized at 30 μW and 0.5 s, respectively. These values remained constant during the entire experiment. The raster scan interval was 1.5 mm and was held constant during the acquisition of each frame; 1024 such points were scanned for the region of interest (ROI). The data were recorded as temporal point-spread functions (TPSF) and the images were reconstructed as fluorescence intensity maps.

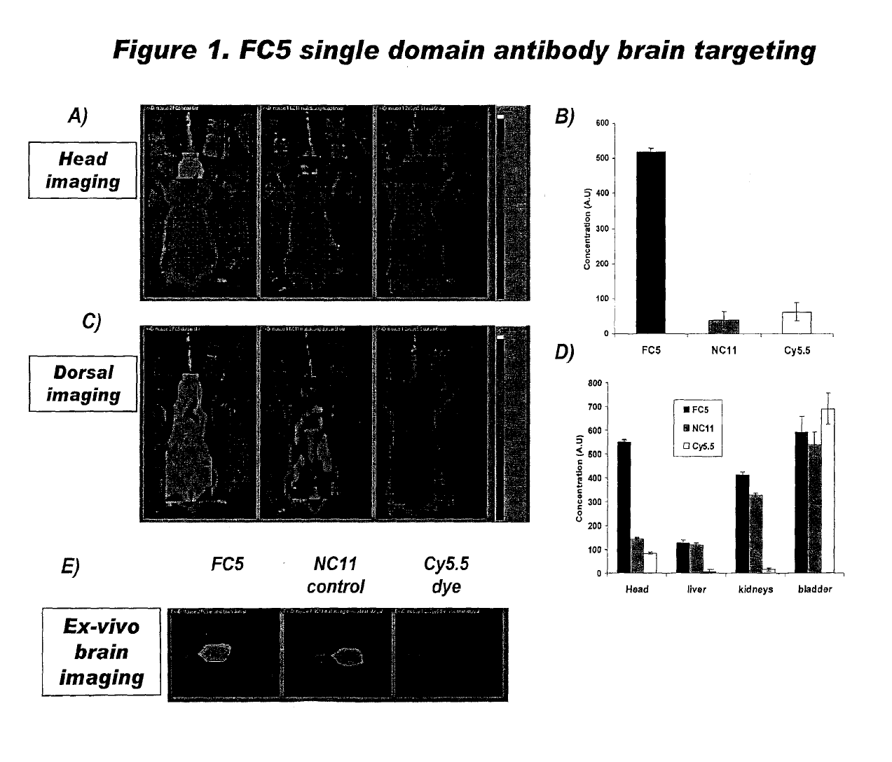

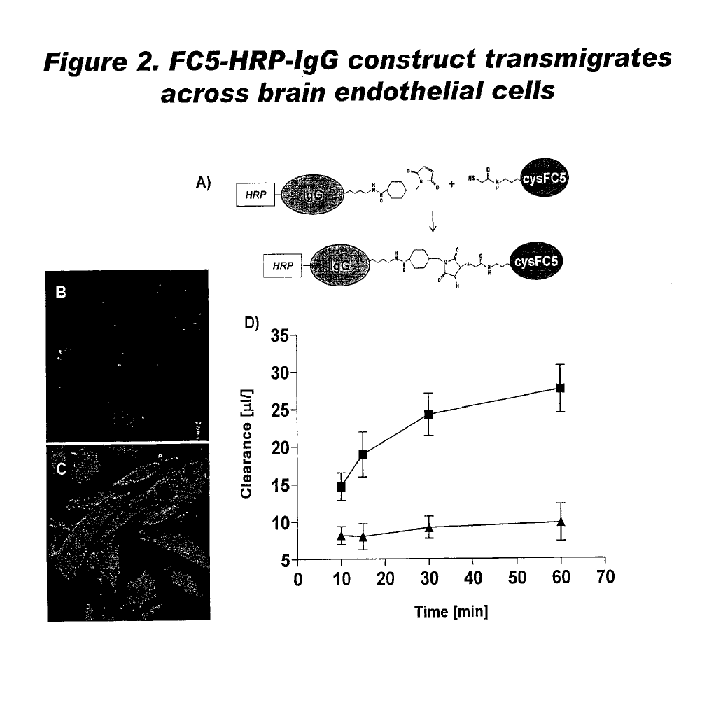

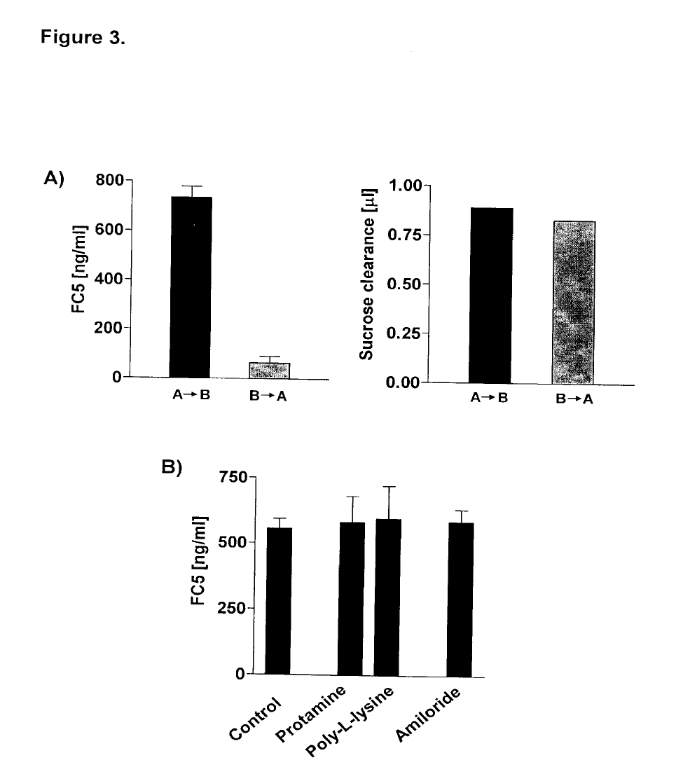

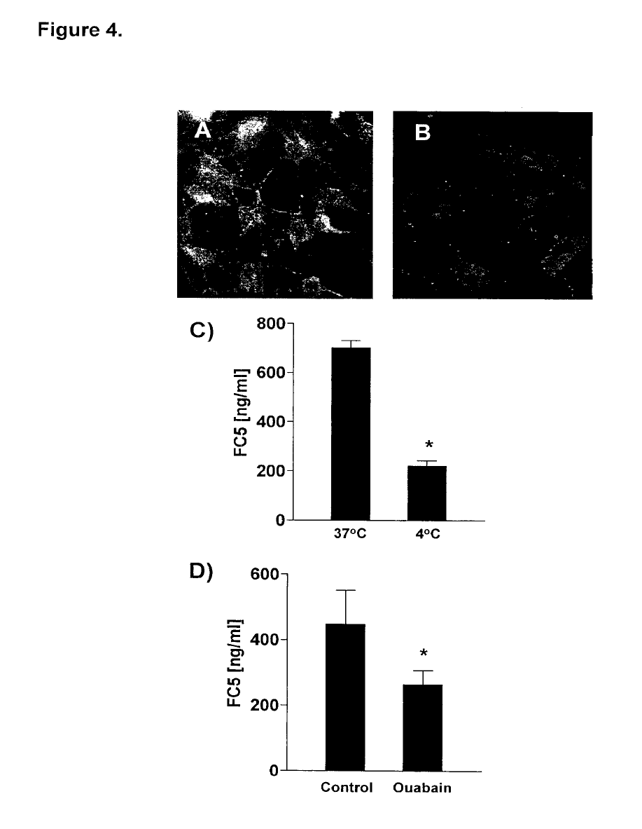

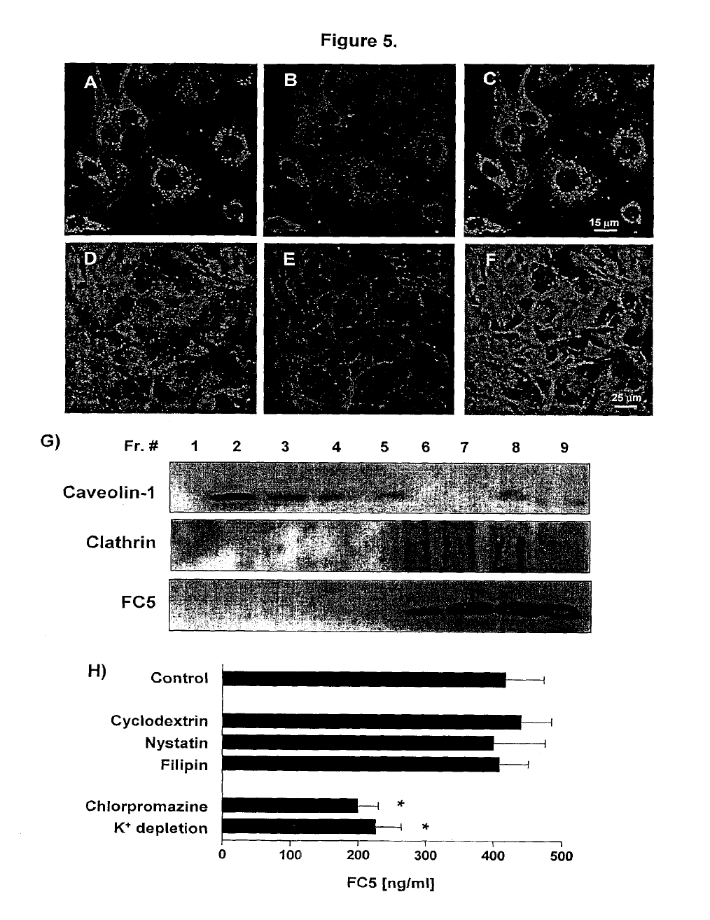

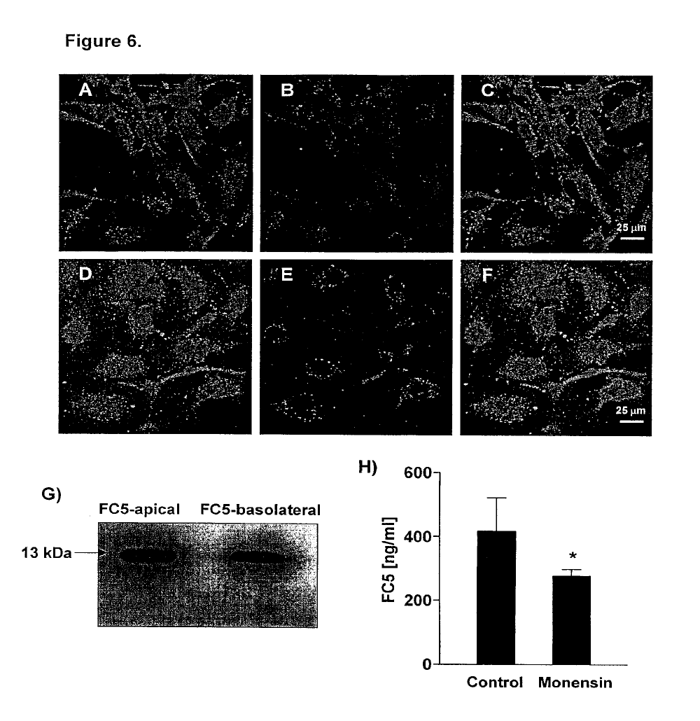

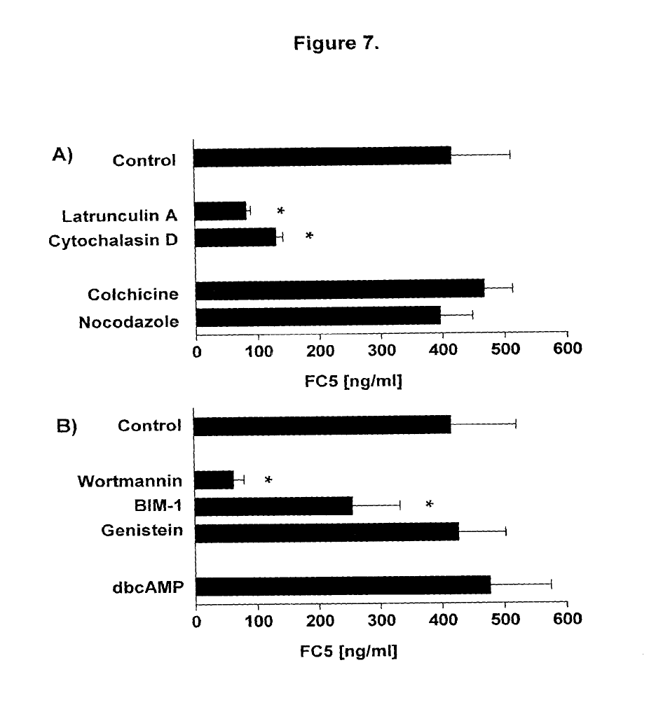

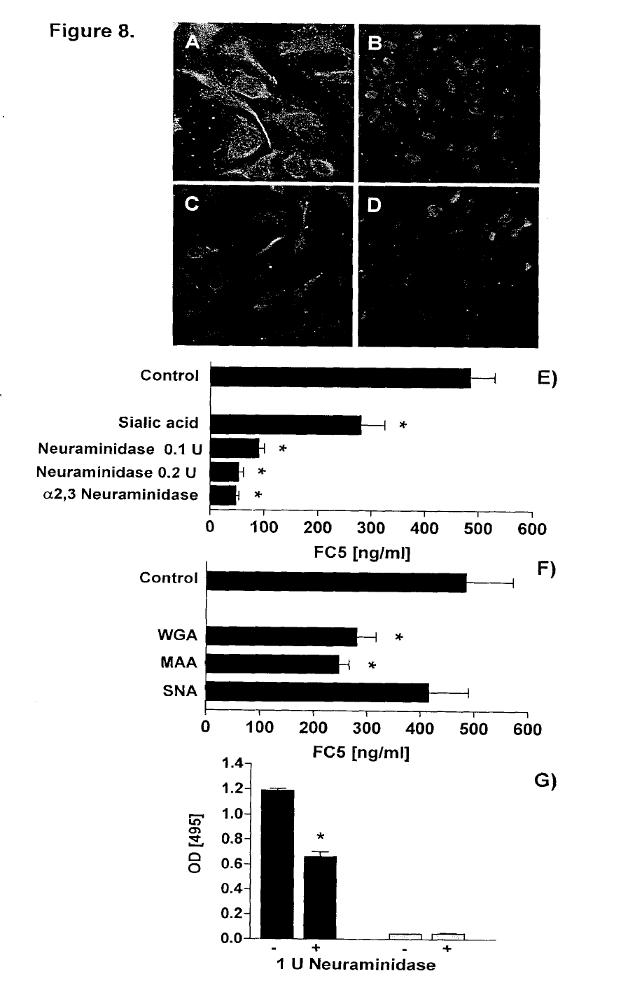

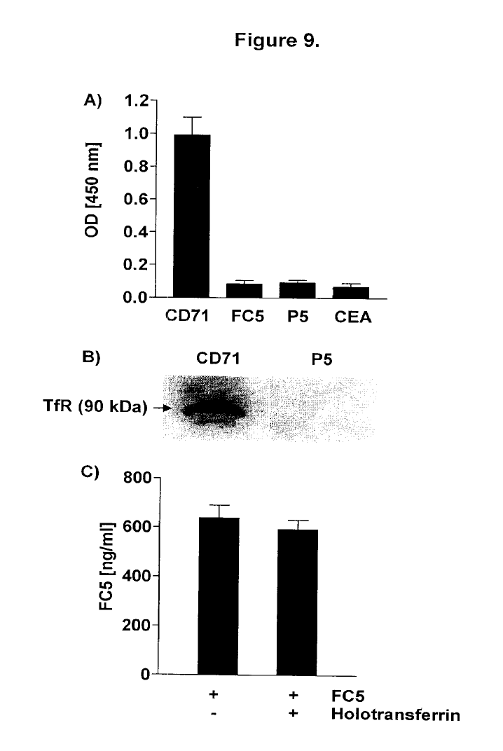

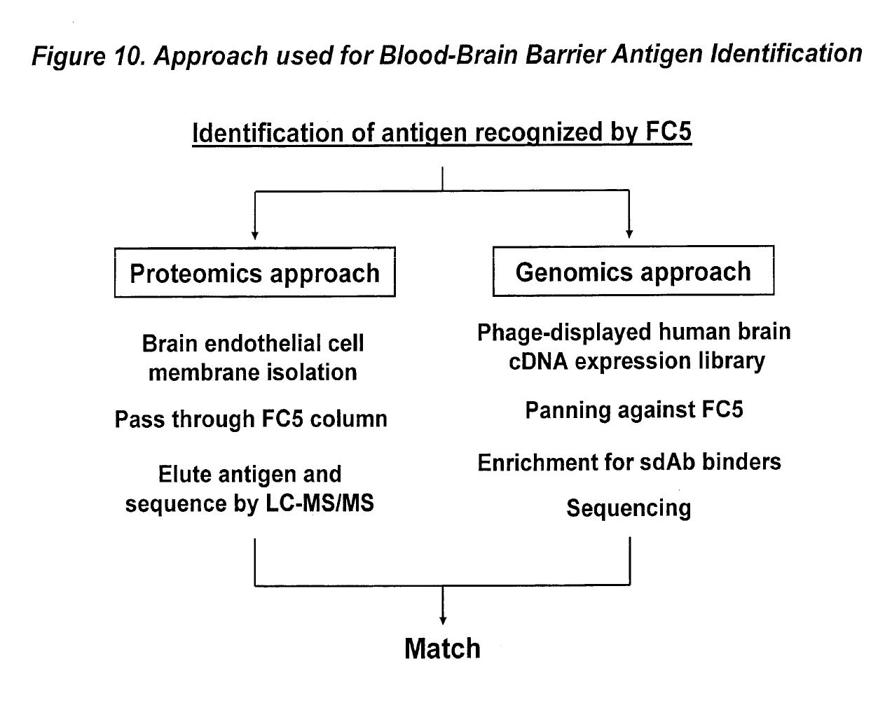

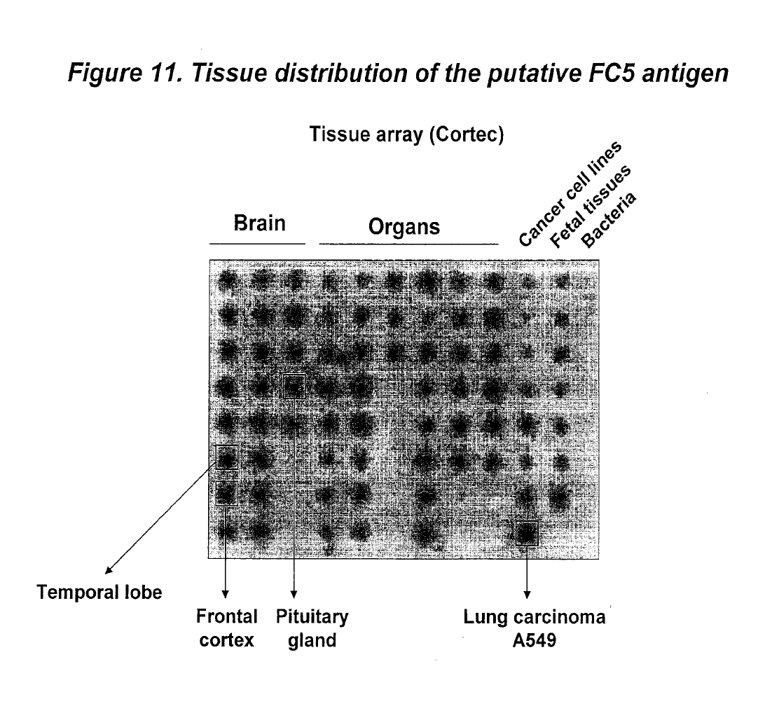

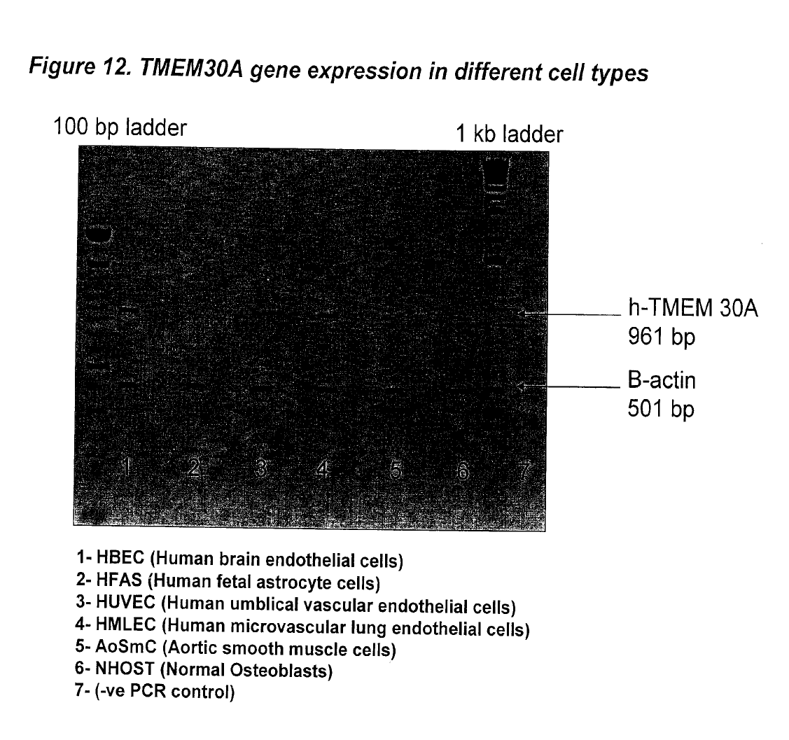

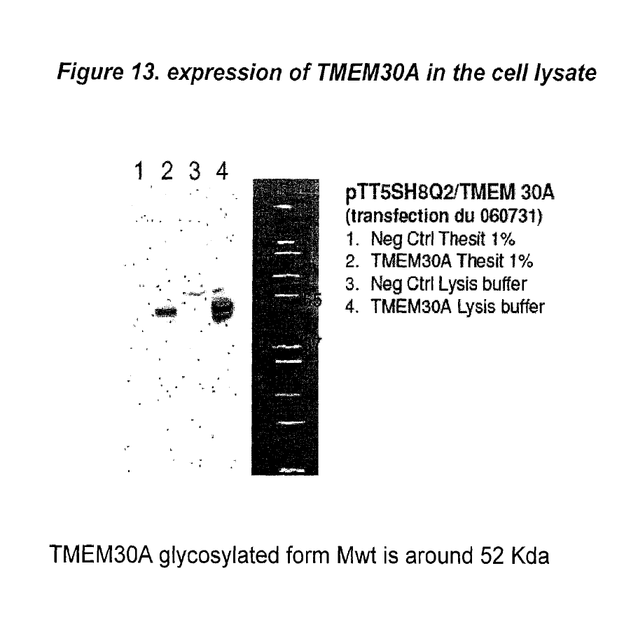

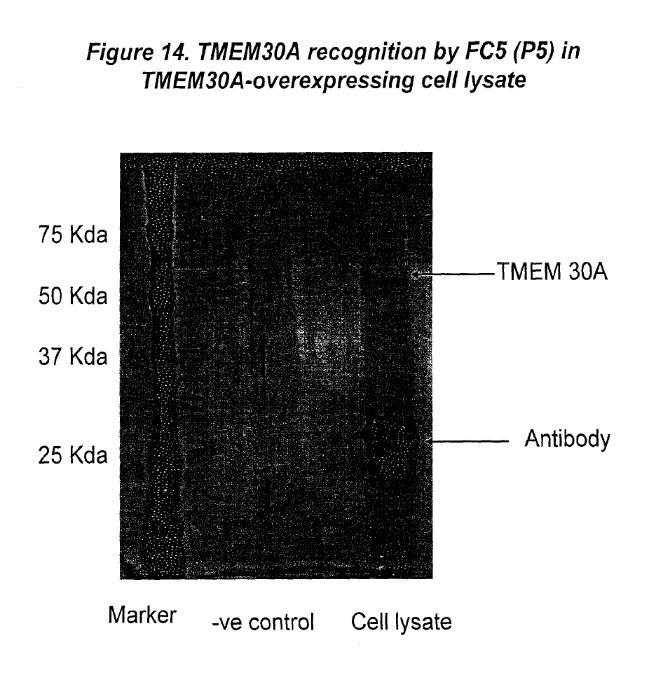

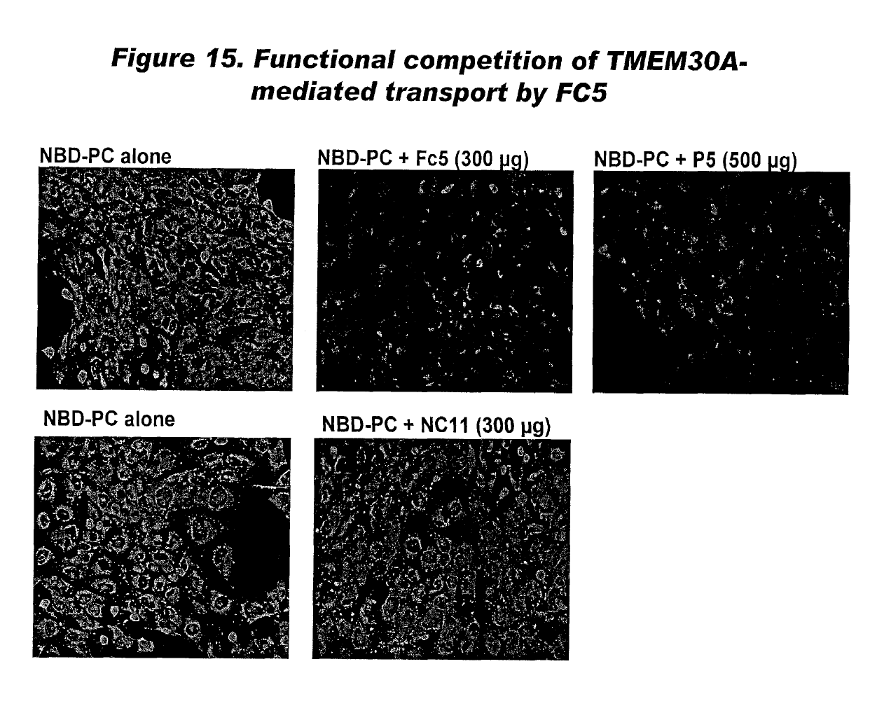

Optical imaging using eXplore Optix small animal imager (670 nm excitation laser) 6 hour after injection showed higher accumulation of the FC5 in the head region compared to the negative control single-domain antibody, NC11, isolated from the same library against different target (FIG. 1). Quantification of the fluorescence concentration using OptiView software in various regions, including head (FIG. 1, B&D) showed a selective accumulation of FC5 in the head. Ex-vivo imaging of brains removed from animals after kill perfusion (FIG. 1E) demonstrate higher fluorescence accumulation in the brain of FC5-injected animals compared to those injected with NC11.

### Example 2

### FC5 is Capable of Carrying ‘Cargo’ Molecules Across the Blood-Brain Barrier Endothelial Cells

Since sdAbs have no available —SH groups for conjugation with therapeutic moieties, FC5 was engineered to express an additional free cysteine. CysFC5 was then conjugated with mouse HRP-IgG (˜190 kDa) using maleimide activation reaction as shown in FIG. 2A. HRP-IgG or HRP-IgG-cysFC5 uptake into human CEC cultures was determined after exposing cells to either construct for 30 min. A significant cellular uptake of IgG-HRP was seen only when the molecule was linked to cysFC5 (FIG. 2 B&C). Similarly, HRP-IgG linked to cysFC5 exhibited a significant transcellular migration to the abluminal chamber of the in vitro BBB model (FIG. 2D) while transport of IgG-HRP alone across human CEC monolayer was negligible (FIG. 2D).

It was demonstrated that only HRP-IgG ‘vectorized’ with FC5 entered human CEC and transmigrated across in vitro BBB, suggesting that sdAbs could successfully shuttle up to 10 times larger molecules into/across target tissues. Using similar chemical linking principles, large molecules of choice with potential therapeutic properties can be attached to cysFC5. Other chemical linker approaches that have been used for whole or single chain antibodies, including biotin-avidin linker, could also be employed with sdAbs providing that appropriate spacers are used to avoid steric hindrance with the antigen binding site. Given the ease with which sdAbs can be genetically engineered, alternative approaches to chemically linking therapeutic molecules are also possible, including chimeric (fusion) proteins

Engineering of BBB-permeable sdAb FC5 to provide free linker moieties, such as that achieved with cysFC5, will enable alternative approaches for their multimeric display in the context of drug carriers. For example, cysFC5 could be conjugated to polymeric components of nanoparticle delivery system or to liposome-based particles using approaches similar to those reported for those reported for IgGs or scFvs. These ‘containers’ vectorized with sdAbs could then be used to deliver drug payloads into the brain, a concept that has already been exploited using ‘classical’ antibodies against few known BBB antigens, including transferrin receptor.

### Example 3

### Mechanisms of FC5 Internalization and Transmigration Across Brain Endothelial Cells

FC5 transmigration across HCEC is polarized and charge-independent FC5 was not toxic to HCEC even at very high concentrations (1 mg/ml). The permeability of [¹⁴C]-sucrose across the in vitro BBB model was not significantly different in the absence or presence of 10 μg/ml FC5 [Pₑ=(0.897±0.11) X10⁻³ and (0.862±0.18)×10⁻³ cm/min, respectively], suggesting that FC5 does not affect the paracellular permeability of HCEC. Transcytosis of FC5 across the in vitro BBB model was polarized: 12-fold higher transport of FC5 from apical-to-basolateral than from basolateral-to-apical chamber was observed in only 30 minutes (FIG. 3A). In contrast, [¹⁴C]-sucrose, a marker for paracellular diffusion, exhibited expected equal (i.e., non-polarized) distribution from apical-to-basolateral and from basolateral-to-apical side of the cellular monolayer (FIG. 3A).

To investigate whether FC5 is internalized and transported by macropinocytosis, FC5 transmigration was tested in the presence of 500 μM amiloride, a compound that inhibits the formation of macropinosomes without affecting coated pits-mediated endocytosis (West et al., 1989). Amiloride had no effect on transendothelial migration of FC5 (FIG. 3B).

The contribution of AME to FC5 transcytosis was assessed because sdAbs are positively charged (the calculated isoelectric point of FC5 is ˜9.23). HCEC were preincubated for 30 minutes with highly cationic protamine sulfate (40 μg/ml) or poly-L-lysine (300 μM), both previously shown to inhibit AME (Sai et al., 1998) prior to assessing FC5 uptake and transport. Neither compound affected FC5 uptake into HCEC (data not shown) nor transport across the in vitro BBB model (FIG. 3B), suggesting that FC5 binding to and transmigration across HCEC is charge-independent.

Surprisingly, wheat germ agglutinin (WGA), tested in these studies for its reported ability to stimulate AME in BBB, significantly inhibited FC5 transmigration providing initial evidence that endothelial glycocalyx might participate in this process through mechanisms other than charge-mediated interactions. This possibility was further explored in studies described later.

### FC5 Transport Across HCEC is Energy-Dependent

To investigate the energy-dependence of FC5 trancytosis, uptake and transport of FC5 were measured at 37° C. and at 4° C. Intracellular FC5 was detected by immunochemistry for c-myc followed by FITC-labeled secondary antibody. FC5 was internalized into HCEC as early as 15 min and was detected in a majority of cells 30 minutes after addition at 37° C. (FIG. 4A). Marked reductions of both intracellular accumulation (FIGS. 4A&B) and trans-endothelial migration (FIG. 2C) of FC5 were observed at 4° C. compared to 37° C. The transport of [¹⁴C]-sucrose across the BBB model was not affected by temperature. A simultaneous inhibition of respiration and glycolytic pathway by exposing HCEC to 5 mM sodium azide (NaN₃) and 5 mM 2-deoxyglucose for 30 min in a glucose-free medium resulted in a near-complete inhibition of FC5 transmigration (FIG. 4C). This treatment has been shown to result in a complete depletion of cellular ATP in other cell types (Ronner et al., 1999). Pretreatment of HCEC with the Na⁺,K⁺-ATPase pump inhibitor, ouabain (1 μM) for 30 minutes also reduced FC5 transport across HCEC by 40% (FIG. 4D).

### FC5 Transcytosis Occurs Via Clathrin-Coated Vesicles

Two major energy-dependent receptor-mediated endocytosis/transcytosis routes for FC5 transmigration, clathrin-coated vesicles and caveolae, were investigated using co-localization studies and endocytosis inhibitors.

Double immunocytochemistry for caveolin-1 and FC5 in HCEC exposed to 5 μg/ml FC5 for 30 minutes showed no co-localization of caveolin-1 immunofluorescence B with FC5 immunofluorescence A (FIG. 5D-F). In contrast, clathrin immunofluorescence E mostly co-localized with that of FC5 D (FIG. 5A-C). Furthermore, after HCEC fractionation by the density gradient centrifugation, FC5 immunoreactivity on Western blot appeared in the same fractions (#7, 8 and 9) as did clathrin immunoreactivity, but was absent from caveolin-1 enriched fractions (#2 and 3) (FIG. 5G).

Uptake and transmigration of FC5 was examined in cells pretreated for 30 minutes with pharmacological inhibitors of clathrin-mediated endocytosis including chlorpromazine (50 μg/ml) and a hypotonic K⁺ depletion buffer (0.14 M NaCl, 2 mM CaCl₂, 1 mg/ml glucose, 20 mM HEPES, pH 7.4 diluted 1:1 with water) or inhibitors of caveolae-mediated endocytosis including filipin (5 μg/ml), nystatin (5 μg/ml) and methyl-β cyclodextrin (5 mM). Chlorpromazine disrupts the recycling of AP-2 from endosomes and prevents the assembly of coated pits on the plasma membrane whereas K⁺ depletion arrests clathrin-coated vesicle formation. Filipin and nystatin bind cholesterol while methyl-β cyclodextrin extracts cholesterol from plasma membrane resulting in disruption of cholesterol-rich caveolae vesicles. None of the caveolae-mediated endocytosis inhibitors tested affected the transmigration of FC5 across in vitro BBB model (FIG. 5H). In contrast, chlorpromazine and K⁺ depletion inhibited the transmigration of FC5 by 52% and 46%, respectively (FIG. 5H).

To investigate intracellular fate of FC5 after endocytosis, colocalization studies were performed with markers of early and late endosomes/lysosomes. FC5 co-localized with the early endosome marker, texas red-conjugated transferrin (FIG. 6A-C) did not co-localize with cathepsin B (FIG. 6D-F), a marker for late endosomes. Transcytosed FC5 collected from the basolateral chamber of the BBB model was indistinguishable from FC5 added to the apical compartment on a Western blot (FIG. 6G), indicating that FC5 bypasses lysosomes and remains intact during transcytosis across HCEC. Un-selected sdAbs from the same library could not be detected in the basolateral chamber of the model (Muruganadam et al., 1997) indicating that FC5 does not pass into basolateral chamber via paracellular transport.

Transport of FC5 was also sensitive to neutralization of intracellular compartments by the cationic ionophore monensin. Monensin breaks down Na⁺ and H⁺ gradients in endosomal and lysosomal compartments, raising the pH of endocytic vesicles from 5.5 to greater than 7 and therefore inhibiting receptor recycling. Monensin (25 μM) inhibited FC5 transcytosis across HCEC by 34% (FIG. 6H) demonstrating that acidified intracellular compartments and recycling of the FC5 putative receptor might be important for maintenance of efficient transendothelial transport.

### Signaling Pathways Involved in FC5 Endocytosis/Transcytosis in HCEC

To determine requirement for cytoskeletal machinery in transcytosis of FC5, HCEC were pre-incubated for 30 minutes with the actin depolymerizing agents, cytochalasin D (0.5 μM) or latrunculin A (0.1 μM), or with the microtubule disrupting agents, nocodazole (20 μM) or colchicine (20 μM). Both cytochalasin D and latrunculin A substantially (70-80%) reduced apical to basolateral transport of FC5 across HCEC (FIG. 7A). In contrast, microtubule-disrupting agents did not interfere with FC5 transcytosis (FIG. 7A).

To determine which signaling pathways modulate transcytosis of FC5, HCEC were pre-incubated for 30 minutes with one of the following modulators: tyrosine kinase inhibitor, genistein (50 μM); protein kinase C(PKC) inhibitor, bisindolyl-maleimide-1 (BIM-1; 5 μM); PI3-kinase inhibitor, wortmannin (0.5 μM); and protein kinase A (PKA) activator, dibutyryl-cAMP (db-cAMP; 500 μM). FC5 transcytosis across HCEC was not affected by either genistein (FIG. 7B) or db-cAMP (FIG. 7B), was reduced by 25% in the presence of PKC inhibitor (FIG. 7B) and was almost completely blocked by PI3 kinase inhibitor (FIG. 7B). None of the pharmacological agents used was toxic to the cells.

### Role of Carbohydrate Epitope(s) in FC5 Transcytosis

The role of endothelial glycocalyx in FC5 transcytosis was indicated by the observation that WGA, a lectin known to stimulate AME in BBB (Banks et al., 1998), inhibited FC5 uptake (FIGS. 8A and 8B) into HCEC.

To test whether proteoglycans, glycoproteins which carry large unbranched polymers composed of 20-200 repeating disaccharide units of sulfated glycosaminoglycan (GAG) chains and are abundantly expressed in CEC, mediate FC5 transcytosis across HCEC, a competition experiments with several known soluble GAGs found on membranes were performed. Pre-incubation of HCEC with heparin sulfate (50 U/ml), chondroitin sulfate A (10 μg/ml) and chondroitin sulfate C (10 μg/ml) did not affect FC5 transcytosis across the BBB in vitro. Similarly, mannan (1 mg/ml) and mannose (50 μM) did not affect FC5 transmigration, suggesting that mannose 6-phosphate/insulin-like growth factor 2 receptor, a multifunctional transmembrane glycoprotein involved in BBB transport in developing brain, was not involved in FC5 internalization.

Since WGA is known to interact with a broad range of sialoconjugates, the importance of sialic acid residues for endo- and transcytosis of FC5 was examined next. HCEC were pre-treated with 200 μM sialic acid, or 0.1-0.2 U of neuraminidase from Vibrio cholerae which sheds all sialic acid from a variety of plasma membrane glycoproteins, or α(2,3) neuraminidase from Salmonella Typhi, that is selective for α(2,3)-linked sialic acid. Both FC5 uptake (FIGS. 8C and 8D) and its transcytosis across HCEC (FIG. 8E) were inhibited by sialic acid and neuraminidase (sialidase). Neuraminidase was especially effective as it reduced FC5 transcytosis by 91% (FIG. 8E). These studies imply that sialic acid is an essential component of the antigenic epitope on HCEC recognized by FC5, since its removal or competition for FC5 binding by exogenous sialic acid interfered with both the uptake and transcytosis of FC5.

The nature of sialoglycoconjugates involved in FC5 transcytosis was examined further by pre-treating cells with three sialic acid-binding lectins: wheat germ agglutinn (WGA; 100 μg/ml) that interacts with a broad range of sialoconjugates, Sambucus nigra agglutinin (SNA; 100 μg/ml) and Maackia amurensis agglutinin (MAA; 100 μg/ml) that recognize α(2,6) and α(2,3) sialylgalactosyl residues, respectively. WGA and MAA inhibited FC5 transcytosis by 40-50% (FIG. 8F), whereas SNA was ineffective (FIG. 8F).

To investigate whether FC5-recognized sialic acid residues are attached to a glycolipid (ganglioside), HCEC cells were fractionated into protein and lipid fractions as described (Wessel and Flugge, 1983). FC5 binding to these fractions in the absence or presence of neuraminidase was examined by ELISA. FC5 binding to HCEC lipid fraction was negligible (FIG. 6G). FC5 also failed to recognize isolated brain gangliosides. In contrast, strong FC5 binding to HCEC protein fraction was reduced by 50% in protein fraction of cell lysates exposed to neuraminidase (FIG. 8G). FC5 did not bind to either protein or lipid fraction of HEK293 cells. Galactosylceramide used as a positive control rendered a strong signal for the lipid fraction detected by O1 anti-galactosylceramide antibody.

### Exclusion of the Transferrin Receptor

Because transferrin receptors are enriched in CEC (Jefferies et al., 1984), are involved in transcytosis across the BBB (Qian et al., 2002), and are highly glycosylated (Hayes et al., 1992), we investigated whether the putative receptor for FC5 is actually the human transferrin receptor. FC5 and its higher avidity pentameric construct P5 (Abulrob et al., 2005) did not bind to immobilized human transferrin receptor in the ELISA assay (FIG. 9A) nor did they recognize the protein on a Western blot (FIG. 9B), in contrast to anti-transferrin receptor antibody CD71 (FIG. 9A,B). In addition, FC5 uptake (data not shown) and transendothelial transport (FIG. 9C) were not reduced in the presence of a 100-fold excess of holo-transferrin.

### DISCUSSION

The collective evidence presented in this study shows that FC5 uptake and transcytosis occur via clathrin-coated vesicles and are dependent on the recognition of neuraminidase-sensitive, α(2,3)-sialo-glycoconjugates. These conclusions were supported by a series of experiments that demonstrated the polarization and temperature and energy-dependence of FC5 transmigration and excluded paracellular diffusion, pore formation and macropinocytosis routes. However, contrary to a common assumption, recent studies on a new class of membrane-penetrating peptides that exhibit charge-mediated BBB selectivity showed that, similar to RME, AME can also be temperature- and energy-dependent (Drin et al., 2003). The failure of AME inhibitors that neutralize negative charge on CEC to reduce transendothelial transport of positively-charged FC5 further suggested RME mechanism. Two major vesicular routes of RME, clathrin-coated pits and caveolae were examined next. Clathrin-coated vesicular pathway of FC5 internalization was indicated by strong co-localization of FC5 with clathrin but not with caveolin immunoreactivity in both intact and fractionated HCEC and by the inhibition of FC5 transcytosis with treatments previously shown to interrupt clathrin-coated vesicle formation. Upon internalization, FC5 was targeted to early endosomes, bypassed late endosomes/lysosomes and was exocytosed into the abluminal compartment without significant intracellular degradation.

The vesicular transcellular transport of FC5 was strongly dependent on the intact actin polymerization. Recent studies have identified several proteins, including Abp1p, Pan1p and cortactin, that functionally link the actin filament assembly with clathrin-coated vesicle internalization.

The complexity of signaling events that control trafficking of clathrin-coated vesicles remains difficult to decipher. FC5 transcytosis was essentially blocked by the PI3-kinase inhibitor, wortmannin, while it was little affected by modulators of other signaling pathways, including PKC-, PKA-, and tyrosine kinase inhibitors. Phosphorylation of inositol lipids by PI3-kinase has been implicated in diverse membrane transport events including clathrin-coated pits pathway. PI3K-C2alpha has been co-purified with a population of clathrin-coated vesicles, whereas proteins involved in the function of these vesicles, including AP-2 and dynamin interact with PI3 kinase. Although PKC and PKA have been implicated in internalization of various receptors, neither appears to be generally required for clathrin-mediated endocytosis. Inhibition of the tyrosine kinase activity of some membrane receptors including the insulin growth factor (IGF) receptor, previously exploited for RME-mediated brain delivery (Zhang et al., 2002), prevents their internalization. The lack of genistein effect on FC5 transcytosis suggested that the receptor recognized by FC5 is likely not a tyrosine kinase.

The surface of brain endothelial cells is covered by a dense layer of complex carbohydrates that participate in cell-cell communication, pathogen recognition/adhesion and interactions with the extracellular matrix (Pries et al., 2000). Studies using various modulators or competitive inhibitors of surface glucoconjugates demonstrated that neuraminidase-sensitive, α(2,3)-sialic acid residues are important for FC5 antigen recognition, FC5 internalization and transcytosis. Sialic acid residues that can be attached to either glycoproteins or gangliosides are abundant in clathrin-coated pits. The major gangliosides expressed in HCEC are GM3 and sialyl paragloboside (LM1). FC5 failed to bind lipids extracted from HCEC or to recognize any of the major brain gangliosides indicating glycoprotein nature of the antigen. Since sialic acid residues are expressed in many tissues, the selectivity of FC5 for brain endothelial cells is likely conferred by a protein component of the antigenic epitope.

The transferrin receptor is brain endothelium enriched, N- and O-glycosylated transmembrane protein with multiple sialic acid residues that undergoes a clathrin-coated vesicle-mediated endocytosis. The antibody against transferrin receptor, OX26, has been used as a vector for brain targeting of biologics and liposomes. FC5 failed to recognize purified human transferrin receptor, and holo-transferrin did not compete with FC5 transcytosis. In agreement with this, desialylated and N-deglycosylated transferrin receptor variants have been shown to exhibit the same transferrin binding and internalization properties as the native transferrin receptor. In addition to the transferrin receptor, other iron-carrying molecules, including melanotransferrin (p97) and lactoferrin, as well as other receptors, including insulin receptor (Zhang et al., 2002) and a low-density lipoprotein receptor (Dehouck et al., 1997) have been identified as potential RME routes for brain delivery. Other studies suggested that receptors specifically up-regulated in pathological conditions, such as TNFβ receptor (Osburg et al., 2002), undergo RME in brain endothelial cells. These proteins have not been specifically excluded as putative antigens recognized by FC5.

In summary, FC5 is a novel single domain antibody that recognizes α(2,3)-sialoglycoprotein expressed on the luminal surface of brain endothelial cells and undergoes actin- and PI3 kinase-dependent transcytosis via clathrin-coated vesicles. FC5 and its derivatives engineered to provide linker moieties (Abulrob et al., 2005) could be developed into brain-targeting vectors for drugs, biologics and nanocarriers. In vivo biodistribution studies (Muruganandam et al., 2001) demonstrated a significant FC5 accumulation in the brain and its rapid elimination via kidneys and liver, typical for other biologics of the similar size. Therefore, improving FC5 pharmacokinetics by strategies such as PEGylation may be necessary for achieving efficient in vivo brain targeting. Nonetheless, BBB-targeting sdAbs combine peptide-like size and high charge-mediated binding to brain endothelium (similar to cell-penetrating Syn-B peptides) (Drin et al., 2003) with the recognition of cell-specific antigens that undergo transendothelial transport, similar to ‘classical’ antibody vectors such as OX26 antibody. Unlike peptides, sdAbs are remarkably resistant to proteases, and, unlike full IgGs, they cannot be exported from the brain via the Fc receptor-mediated efflux system at the BBB. These advantages make sdAbs a versatile alternative to current technologies designed to target drugs and biologics to the brain by exploiting vesicular transendothelial transport.

### Example 4

### Antigen Identification by Panning of Phage-Display Human cDNA Library Against FC5

To identify protein antigen recognized by FC5, a combination of genomics and proteomics methods was used. The strategy is shown schematically in FIG. 10. Genomics approach consisted of panning a phage display library of human brain cDNA (Cortec) against immobilized FC5. After 4 rounds of panning, the most frequent sequence recognized by FC5 was identified—SEQ ID No 1.

The Blast analyses aligned SEQ ID No 1 with the nucleotide sequence 1598-1979 of the Transmembrane protein 30A (synonyms: C6orf67, CDC50A, Cell cycle control protein 50A) nucleotide sequence (Genebank NM_{—}018247). The coding region of the transmembrane domain protein 30A (TMEM30A) is shown as SEQ ID No 2. Splicing variants of coded protein are shown as SEQ ID No 3, SEQ ID No 4, and SEQ ID No 5. Extracellular domain of TMEM30A is shown as SEQ ID No 6. Amino acid sequence of TMEM30A that contain N-glycosylation sites are shown as SEQ ID No 7 and SEQ ID No 8. Sequences in the conserved CDC50 domain of TMEM30A also found with some minor modifications in TMEM30B are shown as SEQ ID No 9-15. It is noted that these sequences are discussed in detail throughout the application.

SEQ ID No 1.
GAA TTT TAT GGA GAA AGG GAT TAC AAG ATG TAT GAG TAT AAT GAC TTG CTA ACC TTT
CAG GAT TCA GAG AAA GAT GAA GAA AGA CCA TAT CTA AAT AAT ACA CTT CAT CAT TTT
CAT GTG TAT AAA TGC TTA AAG TAC CAT CTT TGT TGA GGT GGT TCA TGT ATC CAG TTT
ATC CAG TAC AGT TAT TTG TCA AGC TTA GCT TTG ATT TCA AAG GAC ACG CTT ACC TTG
TCT GGC ATA AGA ATT AAT GCT CAT GTC TGC AGT GGT TGG GTA GGT CCT GCT TAG GAG
AAT TAA AAA ATT CCT CTT TCC GTT TGG TTG AAT GTT GCA GTC AGG AAC CCC AAC TCA
CTT GGA ATG TTT TCA TAT GTA ATC ATT TCC CTT GAA GCT TAT

This sequence was obtained from panning of phage displayed human brain cDNA library against FC5. This sequence aligned with the nucleotide sequence 1598-1979 of TMEM30A nucleotide sequence (genebank NM_{—}018247) and is non-coding.

The nucleotide coding region (141-1226) of of TMEM30A
(Synonyms: Transmembrane protein 30A, TMEM30A, C6orf67, CDC50A,
Cell cycle control protein 50A,
SEQ ID No. 2
atggcgatga actataacgc gaaggatgaa gtggacggtg
ggcccccgtg tgctccgggg ggcaccgcga agactcggag accggataac acggccttca
aacagcaacg gctgccagct tggcagccca tccttacggc tggcacggtg ctacctattt
tcttcatcat cggtctcatc ttcattccca tcggcattgg catttttgtc acctccaaca
acatccgcga gatcgagatt gattataccg gaacagagcc ttccagtccc tgtaataaat
gtttatctcc ggatgtgaca ccttgctttt gtaccattaa cttcacactg gaaaagtcat
ttgagggcaa cgtgtttatg tattatggac tgtctaattt ctatcaaaac catcgtcgtt
acgtgaaatc tcgagatgat agtcaactaa atggagattc tagtgctttg cttaatccca
gtaaggaatg tgaaccttat cgaagaaatg aagacaaacc aattgctcct tgtggagcta
ttgccaacag catgtttaat gatacattag aattgtttct cattggcaat gattcttatc
ctatacctat cgctttgaaa aagaaaggta ttgcttggtg gacagataaa aatgtgaaat
tcagaaatcc ccctggagga gacaacctgg aagaacgatt taaaggtaca acaaagcctg
tgaactggct taaaccagtt tacatgctgg attctgaccc agataataat ggattcataa
atgaggattt tattgtttgg atgcgtactg cagcattacc tacttttcgc aagttgtatc
gtcttataga aaggaaaagt gatttacatc caacattacc agctggccga tactctttga
atgtcacata caattaccct gtacattatt ttgatggacg aaaacggatg atcttgagca
ctatttcatg gatgggagga aaaaatccat ttttggggat tgcttacatc gctgttggat
ccatctcctt ccttctggga gttgtactgc tagtaattaa tcataaatat agaaacagta
gtaatacagc tgacattacc atttaatttt

Coding region of TMEM30A gene encodes 3 splicing variants of TMEM30A protein. Amino acid sequences of these three isoforms are given below:

1. Isoform 1:
>gi|8922720|ref|NP_060717.1|transmembrane
protein 30A [Homo sapiens]
SEQ ID No. 3
MAMNYNAKDEVDGGPPCAPGGTAKTRRPDNTAFKQQRLPAWQPILTAGT
VLPIFFIIGLIFIPIGIGIFVTSNNIREIEIDYTGTEPSSPCNKCLSPD
VTPCFCTINFTLEKSFEGNVFMYYGLSNFYQNHRRYVKSRDDSQLNGDS
SALLNPSKECEPYRRNEDKPIAPCGAIANSMFNDTLELFLIGNDSYPIP
IALKKKGIAWWTDKNVKFRNPPGGDNLEERFKGTTKPVNWLKPVYMLDS
DPDNNGFINEDFIVWMRTAALPTFRKLYRLIERKSDLHPTLPAGRYSLN
VTYNYPVHYFDGRKRMILSTISWMGGKNPFLGIAYIAVGSISFLLGVVL
LVINHKYRNSSNTADITI
2. Isoform 2:
>sp_vs|Q9NV96-2|Q9NV96 Isoform 2 of Q9NV96
SEQ ID No. 4
MAMNYNAKDEVDGGPPCAPGGTAKTRRPDNTAFKQQRLPAWQPILTAGT
VLPIFFIIGLIFIPIGIGIFVTSNNIREIEGNVFMYYGLSNFYQNHRRY
VKSRDDSQLNGDSSALLNPSKECEPYRRNEDKPIAPCGAIANSMFNDTL
ELFLIGNDSYPIPIALKKKGIAWWTDKNVKFRNPPGGDNLEERFKGTTK
PVNWLKPVYMLDSDPDNNGFINEDFIVWMRTAALPTFRKLYRLIERKSD
LHPTLPAGRYSLNVTYNYPVHYFDGRKRMILSTISWMGGKNPFLGIAYI
AVGSISFLLGVVLLVINHKYRNSSNTADITI
Isoform 2 is missing amino acids 79-114.
3. Isoform 3:
>sp_vs|Q9NV96-3|Q9NV96 Isoform 3 of Q9NV96
SEQ ID No. 5
MYYGLSNFYQNHRRYVKSRDDSQLNGDSSALLNPSKECEPYRRNEDKPI
APCGAIANSMFNDTLELFLIGNDSYPIPIALKKKGIAWWTDKNVKFRNP
PGGDNLEERFKGTTKPVNWLKPVYMLDSDPDNNGFINEDFIVWMRTAAL
PTFRKLYRLIERKSDLHPTLPAGRYSLNVTYNYPVHYFDGRKRMILSTI
SWMGGKNPFLGIAYIAVGSISFLLGVVLLVINHKYRNSSNTADITI
Isoform 3 is missing amino acids 1-119.
The extracellular domain of TMEM30A
contains amino acids 67-323
SEQ ID No 6
GIFVTSNNIREIEIDYTGTEPSSPCNKCLSPDVTPCFCTINFTLEKSFE
GNVFMYYGLSNFYQNHRRYVKSRDDSQLNGDSSALLNPSKECEPYRRNE
DKPIAPCGAIANSMFNDTLELFLIGNDSYPIPIALKKKGIAWWTDKNVK
FRNPPGGDNLEERFKGTTKPVNWLKPVYMLDSDPDNNGFINEDFIVWM
RTAALPTFRKLYRLIERKSDLHPTLPAGRYSLNVTYNYPVHYFDGRKRM
ILSTISWMGGKNP

Amino acid sequence of TMEM30A that contain N-glycosylation sites:

SEQ ID No 7.
RRNEDKPIAPCGAIANSMFNDTLELFLIGNDSYPIPIALK
(found in TMEM30A residues 160-200).
SEQ ID No 8.
RRNEDKPIAPCGAIANSMFNDTLELFLIGNDSYPIPIALK
KKGIAWWTDKNVKFRNPPGGDNLEERFKGT
TKPVNWLKPVYMLDSDPDNNGFINEDFIVWMRTAALPTFR
KLYRLIERKSDLHPTLPAGRYSLNVTYNYP
(found in TMEM30A residues 160-300).
Residues susceptible to
N-glycosylation: 180, 190, 294.

Sequences in the conserved CDC50 domain of TMEM30A also found with some minor modifications in TMEM30B.

SEQ ID No 9
NFYQNHRRYVKSRDDSQL
(found in TMEM30A residues 126-144 and
found in TMEM3OB residues 115-133).
SEQ ID No 10
APCGAIANSMF
(found in TMEM30A residues 169-179)
SEQ ID No 11
APCGAIANSLF
(found in TMEM3OB residues 160-170)
SEQ ID No 12
DFIVWMRTAALPT
(found in TMEM30A residues 256-269)
SEQ ID No 13
DFVVWMRTAALPT
(found in TMEM3OB residues 249-262)
SEQ ID No 14
MGGKNPFLGIAYIAVG
(found in TMEM30A residues 256-269)
SEQ ID No 15
MGGKNPFLGIAYLVVG
(found in TMEM3OB residues 249-262)

### Tissue Distribution of FC5 Antigen

To analyze tissue distribution of putative FC5 antigen, Cortec tissue microarray displaying tissue extracts from various organs, various brain regions and various cells lines. Tissue microarray was probed with TMEM30A primers, and TMEM30A binding was detected by southern blotting. FIG. 11 shows high reactivity of FC5 (antigen abundance) in various brain regions and lung carcinoma cells.

### Expression of TMEM30A Gene in the Brain

TMEM30A gene expression in different cell lines was tested using RT-PCR using forward 5′GAAGACTCGGAGACCGGATAACAC′3 (SEQ ID No. 16) and reverse 5′ CAGTACAACTCCCAGAAGGAAGGAG′3 (SEQ ID No. 17). FIG. 12 shows the high expression of TMEM30A in human brain endothelial cells (HBEC) and low expression in human fetal asotrcytes. Human umbilical cord vascular endothelial cells (HUVEC) and human lung microvascular endothelial cells (HMLEC) also showed TMEM30A gene expression.

### Example 5

### Antigen Identification by Proteomics

The antigen identification by proteomics was done by: a) extracting plasma membrane of brain endothelial cells (containing the antigen); b) passing the extract through the FC5 or negative control antibody, NC11—bound nickel microspin column; c) collecting the eluates from columns, treatment or not with 0.2 U neauramindase enzyme (from Vibrio cholera, Sigma) and analysing them by mass spectrometry. The approach is described below:

### Plasma Membrane Protein Extraction:

Immortalized rat brain endothelial cells (SV-ARBEC) were plated and grown in 160 cm² Petrie dishes for about one week. Cells were fed by full media change after 4 days. When the cells reached a confluent state, the plasma membrane protein was extracted. Eight 160 cm² Petrie dishes were used. Cells were placed on ice, washed 1× with 30 ml PBS and twice with 10 ml Buffer A (0.25M sucrose, 1 mM EDTA, 20 mM tricine, pH 7.8). 5 ml of Buffer A⁺ (Buffer A plus 1:1000 of inhibitor cocktail form Sigma) was added and cells were scraped off. Cells were then collected in two 50 ml falcon tube. (4 dishes/tube) and spun down at 1400×g for 5 minutes at 4′C. Cells pellets were resuspended in 1 ml Buffer A⁺. Both resuspended pellets were then pooled together and homogenized using a glass tube and Teflon pestle (20 strokes). The homogenate was transferred to two 2 ml centrifuge tube and spun at 1000×g for 10 min at 4 C. The supernatant was collected. The pellet was resuspended in 2 ml Buffer A⁺ and then homogenized. The plasma membrane was overlaid over 20 ml of 30% percoll and spun at 83000×g for 30 min at 4′C. The plasma membrane sample was collected and resuspended in 5 ml of PBS⁺ and spun at 118000×g for 1 h at 4′C. Protein concentration was measured using the BCA kit (Pierce). Sample was aliquoted and frozen at −80 C.

### Antibody Loaded Column for Antigen Identification:

Columns from Amersham microspin His purification module was used to bind the antibodies. Briefly, columns were incubated with 200 μg of FC5, NC11 or simply PBS for 1 h with inversion at RT. Columns were spun at 735×g for 1 min and then washed once with 500 ul PNI₂₀ and twice with 500 ul PBS. 300 μg of plasma membrane protein was incubated in each column for 3.5 hr at 4′C with inversion followed by a 30 min incubation at RT with inversion. Columns were then spun at 735×g for 1 min and then washed 4× with 500 ul PNI₂₀ with centrifugation at 735×g for 1 min between each wash. Proteins were eluted by incubating the columns with 200 ul PNI₄₀₀ for 15 min at RT with inversion and spinning at 735×g for 1 min. the proteins eluted from each sample protein was treated or not with 0.2 U neuramindase for 1 h.

### Trypsin Digestion

Each pull-down sample (FC5, NC11, PBS) was precipitated by adding 10-volume of cold acetone and incubated at −20° C. for >12 h. Proteins were pellet by centrifugation at 5000×g for 5 min and dissolved in 50 μL denaturing buffer (50 mM Tris-HCl, pH 8.5, 0.1% SDS, 4 mM DTT). Proteins were boiled for 15 min to denature and cooled for 2 min. To each sample, 5 μg of trypsin (Promega, cat # V5280) was added and samples were incubated at 37° C. for >12 h.

### Purification on Cation Exchange (CE) Column

Each sample was diluted to 2 mL with CE load buffer (10 mM KH₂PO₄, pH 3.0, 25% acetonitrile) and pH was confirmed to be <3.3. Samples were purified on a cation exchange column (POROS® 50 HS, 50-μm particle size 4.0 mm×15 mm, Applied Biosystems, cat #4326695) as per manufacturer's protocol.

### Mass Spectrometry and Database Searching

A hybrid quadrupole time-of-flight MS (Q-TOF™ Ultima, Waters, Millford, Mass., USA) with an electrospray ionization source (ESI) and an online reverse phase nanoflow liquid chromatography column (nanoLC, 0.3 mm×15 cm PepMap C18 capillary column, Dionex/LC-Packings, San Francisco, Calif., USA) was used for all analyses. The gradient of the nanoLC column used was 5-95% acetonitrile 0.2% formic acid in 50 min, 0.35 μL/min supplied by a CapLC HPLC pump (Waters). Analysis of each sample was done in two steps. In the first step, 5% of sample was analyzed by nanoLC-MS in a survey (MS-only) mode to quantify the intensity of all the peptides present in each sample. Interesting peptides were determined as described in the “quantitative data analysis” section and were included in a “target list.” In the second step, each sample was re-injected (5%) into the mass spectrometer and only the peptides included in the target list were sequenced in a nanoLC-MS/MS mode. MS/MS spectra were obtained only on 2+, 3+, and 4+ ions. These were then submitted to PEAKS search engine (Bioinformatics Solutions Inc., Ontario, Canada) to search against a NCBI nonredundant, trypsin-digested (allowing 2 missed cleavage) human database.

### Quantitative Data Analysis Using MatchRx Software

From the nanoLC-MS raw data of each sample, peak intensities corresponding to the abundance of each peptide was extracted as described earlier (Haqqani et al, FASEB J. 2005 November; 19:1809-21). Peptide intensities were quantitatively compared among all samples using MatchRx software. Peptides present in FC5 pull downs but absent in NC11 and PBS pull down were of interest. Peptides identified by proteomics eluted from FC5 but not to NC11 antibody column are:

| | |
|---|---|
| [symbol] , | (SEQ ID No. 18) |
| [symbol] , | (SEQ ID No. 19) |
| [symbol] , | (SEQ ID No. 20) |
| [symbol] | (SEQ ID No. 21) |

All these peptides belong to TMEM30A protein

### Example 6

### TMEM30A Expression and Recognition by FC5

The TMEM30A protein was next cloned and expressed. The recognition of TMEM30A by FC5 in cell lysates of TMEM30A-expressing cells was used to confirm specific recognition of TMEM30A by FC5.
Cloning Human TMEM30A Gene into pTT5SH8Q2 Vector for His-Tagged Protein Purification in Mammalian Cells.
The pTT5SH8Q2 vector harboring the C-terminal His6 tag was used for cloning TMEM30A gene. The primers used for PCR the coding region for the cloning:

```
TMEM30A forward:
(SEQ ID No. 22)
5′ T CTC GAA TTC ATG GCG ATG AAC TAT AAC GCG 3′
                      EcoRI
TMEM30A reverse:
(SEQ ID No. 23)
5′ T CTC ACC GGT AAT GGT* AAT GTC AGC TGT ATT 3′
                    Agel
```

Plasmids were amplified using the E. coli DH5a strain grown in CiculeGrow broth supplemented with ampicillin (100 μg/ml) and purified using Maxi/Giga plasmid purification kits (Qiagen).

Sequencing was confirmed using the following primers:

| | |
|---|---|
| TMEM30A-SP1 | |
| 5′ TCT CGA TCT CGC GGA TGC 3′ | (SEQ ID No. 24) |
| TMEM30A-SP2 | |
| 5′ CAT CCA ACA TTA CCA GCT 3′ | (SEQ ID No. 25) |
| TMEM30A-SP3 | |
| 5′ CGG ATG ATC TTG AGC ACT 3′ | (SEQ ID No. 26) |

DNA concentration was measured by UV absorbance at 260 nm in 50 mM Tris-HCL pH 8.0.

### Production of TMEM30A Protein

The human embryonic kidney 293 cell line stably expressing Epstein-Barr virus Nuclear Antigen-1 (293E) was grown as suspension culture in low-calcium-SFM (LCSFM, Invitrogen, Grand Island, N.Y.) supplemented with 0.1% Pluronic F-68, 1% bovine calf serum (BCS), 50 μg/ml Geneticin G418, and 10 mM Hepes. The serum-free cell line HEK293 SFE (293SFE) was also used in TMEM30A production. These cells were grown in LC-SFM supplemented with 0.5% of GPN3 as described previously (Pham et al., 2003). All cell passages were routinely done in 125-ml Erlenmeyer flasks containing 20 ml of culture medium. The 293SFE cells were maintained at the exponential phase in suspension in culture flasks containing LC-SFMLB, 10 μg/mL of Geneticin and 10 mM Hepes. The culture flasks were shaken at 110 rpm at 37 C in a humidified, 5% CO₂ atmosphere.

### Expression of TMEM30A in the Cell Lysate.

As shown in FIG. 13, TMEM30A was extracted from the cells using 1% Thesit and deoxycholate. Anti-histidine antibody was used for detection. The expected Mwt of TMEM30A is 40 Kda and the higher protein molecular weight size of around 50 Kda is due to glycosylation.

Interaction of TMEM30A with FC5 Investigated by Immunoprecipitation

To study the interaction of TMEM30A with FC5, 100 μg of supernatant cell lysate from HEK293 that transformed to express TMEM30A were initially pre-cleared by incubation with 50 μl protein A sepharose (50% slurry) for 2 h at 4 degrees with gentle rocking, spin for 4 min at 500 g. Multimeric form of FC5 was used with improved avidity (engineered Pentameric FC5) (25 μg) was added to the cleared supernatant and incubated overnight at 4 degrees. Protein A sepharose (50 μl, 50% slurry) was added to the immunobound lysate and incubated for 2 h at 4 degrees. The immunocomplex was then washed 5 times with ice cold PBS. The slurry was then boiled in laemmeli buffer for 5 min to dissociate the bound protein and centrifuged for 1 min at 14 000 g to collect the immunoprecipitated proteins. Immunoprecipitated proteins were separated on 12% SDS-acrylamide gel and then silver stained to visualize the bands.

As shown in FIG. 14 the pentameric FC5 immunoprecipitated only a band at molecular weight of around 50 identical in size to the protein size observed in FIG. 13. Cells that were not incubated with FC5 pentamer didn't immunoprecipitate TMEM30A.

### Example 7

### Functional Competition of TMEM30A Mediated Transport with FC5

Rat brain endothelial cells were cultured on coverlips for 3 days and then treated with 1-Palmitoyl-2-[6-[(7-nitro-2-1,3-benzoxadiazol-4-yl)amino]hexanoyl]-sn-Glycero-3-Phosphocholine (16:0-06:0 NBD PC) purchased from Avanti lipids (dissolved in DMSO) in the presence or absence of FC5, or pentameric FC5 (P5), or negative control antibody (NC11) for 30 min at 37 C. Cells were then extensively washed and fixed with 4% formaldehyde and then treated with Dako Fluorescent Mounting Medium spiked with DAPI (1:2000 from 2 mg/mL stock). All images were acquired using Axiovert 200 and following settings: 20× objective, DNA-DAPI (blue) 85 msec, NBD-FITC(green) 250 msec.

Results shown in FIG. 15 demonstrates that FC5 and its pentameric form P5 compete with TMEM30A physiological function measured by reduction in internalization of NBD-phosphatidylcholine (NBD-PC). In contrast, negative control antibody NC11 didn't inhibit the internalization of NBD-PC.

### Materials and Methods

### Materials

Cell culture plastics were obtained from Becton Dickinson (Mississauga, ON). Dulbecco's modified Eagle's medium was purchased from Invitrogen (Carlsbad, Calif.), FBS from HyClone (Logan, Utah), human serum from Wisent Inc. (Montreal, QC), and endothelial cell growth supplement from Collaborative Biomedical Products (Bedford, Mass.). Antibodies were obtained from the following sources: anti-c-Myc-peroxidase antibody from Roche (Indianapolis, Ind., USA), anti-caveolin and anti-clathrin antibodies from Santa Cruz Biotechnology (Santa Cruz, Calif.), FITC-conjugated anti-mouse and Alexa 568 conjugated anti-rabbit secondary antibodies from Molecular Probes (Eugene, Oreg., USA), Texas-red conjugated transferrin and calcein-AM were purchased from Molecular Probes (Eugene, Oreg., USA). Monensin and bisindolyl-maleimide-1 (BIM) were from Calbiochem (San Diego, Calif., USA). Optiprep was purchased from Accurate Chemical and Scientific Corp (Westbury, N.Y., USA). Purified human transferrin receptor and monoclonal anti-CD71 (anti-transferrin receptor) antibody were purchased from Research Diagnostics Inc (Flanders, N.J., USA). [¹⁴C]-sucrose was purchased from Perkin Elmer (Boston, Mass., USA). Tetramethylbenzidine (TMB)/hydrogen peroxide substrate system was procured from R&D systems (Minneapolis, Minn.). EZ link sulfo-NHS-LC-LC-biotin and bicinchoninic acid assay (BCA) were purchased from Pierce Biotechnology (Rockford, Ill., USA). All other chemicals were from Sigma (St Louis, Mo., USA).

FC5 sdAb Cloning, Expression and Purification

FC5 is a variable domain (V_{H}H) of the llama heavy chain antibody with encoding mRNA and amino acid sequences deposited in the GenBank (No. AF441486 and No. AAL58846, respectively). DNA encoding FC5 was cloned into the BbsI/BamHI sites of plasmid pSJF2 to generate expression vector for FC5. The DNA constructs were confirmed by nucleotide sequencing on 373A DNA Sequencer Stretch (PE Applied Biosystems) using primers fdTGIII, 5′-GTGAAAAAATTATTATTATTCGCAATTCCT-3′ (SEQ ID No. 27) and 96GIII, 5′-CCCTCATAGTTAGCGTAACG-3′ (SEQ ID No. 28). The FC5 was expressed in fusion with His₅ and c-myc tags to allow for purification by immobilized metal affinity chromatography using HiTrap Chelating™ column and for detection by immunochemistry, respectively. Single clones of recombinant antibody-expressing bacteria E. coli strain TG1 were used to inoculate 100 ml of M9 medium containing 100 μg/ml of ampicillin, and the culture was shaken overnight at 200 rpm at 37° C. The grown cells (25 ml) were transferred into 1 L of M9 medium (0.2% glucose, 0.6% Na₂HPO₄, 0.3% KH₂PO₄, 0.1% NH4Cl, 0.05% NaCl, 1 mM MgCl₂, 0.1 mM CaCl₂) supplemented with 5 μg/ml of vitamin B1, 0.4% casamino acid, and 100 μg/ml of ampicillin. The cell culture was shaken at room temperature for 24 hours at 200 rpm and subsequently supplemented with 100 ml of 10× induction medium Terrific Broth containing 12% Tryptone, 24% yeast extract, and 4% glycerol. Protein expression was induced by adding isopropyl-β-D-thiogalactopyranoside (IPTG; 1 mM). After induction, the culture was shaken for an additional 72 hours at 25° C., and the periplasmic fraction was extracted by the osmotic shock method (Anand et al., 1991). The FC5 fragments were purified by immobilized metal-affinity chromatography using HiTrap Chelating column (Amersham Pharmacia Biotech; Piscataway, N.J.). FC5 produced was eluted in 10 mM HEPES buffer, 500 mM NaCl, pH 7.0, with a 10-500 mM imidazole gradient and peak fractions were extensively dialyzed against 10 mM HEPES buffer, 150 mM NaCl, 3.4 mM EDTA, pH 7.4. The molecular weight of FC5 is 13.2 kDa and that of FC5 fusion protein with c-myc and His₅ tags is 15.2 kDa.

Cloning and Purification of cysFC5

FC5 was engineered to add additional free cysteine that can be used for conjugation with drugs and carriers. DNA encoding sdAb FC5 was cloned into the BbsI/BamHI sites of plasmid pSJF2 to generate expression vector for monomeric FC5. cysFC5 gene was generated from FC5 template by a standard PCR using a forward primer that added a cysteine immediately after the His₅ ‘purification’ tag codons. cysFC5 gene was subsequently cloned into pSJF2 using standard cloning techniques. The integrity of the cloned construct was confirmed by nucleotide sequencing on 373A DNA Sequencer Stretch (PE Applied Biosystems, Streetsville, ON). cysFC5 was expressed in bacteria E. coli strain TG1 and purified by immobilised metal affinity chromatography (IMAC). The eluted fractions homogenous for cysFC5 as judged by SDS-PAGE were pooled and extensively dialyzed against 10 mM HEPES buffer, 150 mM NaCl, 3.4 mM EDTA, pH 7.4. Protein concentrations were determined by the bicinchoninic acid assay (BCA). To assure complete reduction of the engineered free cysteine without compromising the conserved Cys22-Cys92 internal disulfide bonds, the cysFC5 was exposed to 50 mM Tris (2-Carboxyethyl) Phosphine Hydrochloride containing 5 mM EDTA in PBS overnight at 4° C. followed by rapid separation on G-25 sephadex columns prior to conjugation. These conditions did not compromise antigen binding activity of cysFC5 determined by intact cellular uptake and transmigration across CEC monolayers.

### Conjugation of HRP-IgG to CysFC5

Cross linking between the horseradish peroxidase (HRP)-tagged mouse IgG and cysFC5 was achieved using sulphosuccinimidyl-4-(N-maleimidomethyl)cyclohexane-1-carboxylate (sulfo-SMCC) as cross linking agent. Sulfo-SMCC builds a bridge between an amine (—NH₂) functional group on the HRP-IgG and a sulfahydryl (—SH) group on the cysFC5 sdAb. First, HRP-IgG was maleimide-activated by incubation with a 10 molar excess of sulfo-SMCC solution in PBS for 30 min at room temperature. Maleimide reagent was removed by G-25 sephadex columns (Roche Biochemicals, Indianapolis, Ind.). Maleimide-activated HRP-IgG was cross linked with reduced cysFC5 by mixing 5:1 molar ratio at room temperature for 1 h.

### Cell Culture

Primary human cerebromicrovascular endothelial cell (HCEC) cultures were isolated from human temporal cortex removed surgically from perifocal areas of brain affected by idiopathic epilepsy. Cells were dissociated, cultured and characterized as previously described in detail (Stanimirovic et al., 1996; Muruganandam et al., 1997). The morphological, phenotypic, biochemical and functional characteristics of these HCEC cultures have been described previously (Stanimirovic et al., 1996; Muruganandam et al., 1997). Passages 2-6 of HCEC were used for the experiments in this study.

Cell viability in the presence of FC5 and various pharmacological agents was assessed by the vital dye calcein-AM release assay as described previously (Wang et al., 1998).

The uptake of FC5 into HCEC was tested 15-90 minutes after adding 5 μg/ml of FC5 in the absence or presence of various pharmacological modulators of endocytosis. To visualize the intracellular distribution of FC5, cells were fixed, permeabilized and probed with the anti-c-myc antibody (1:100; 1 hour) followed by incubation with FITC-labeled anti-mouse IgG (1:250; 1 hour).

### Transport Across the In Vitro Blood Brain Barrier Model

HCEC (80,000 cells/membrane) were seeded on a 0.5% gelatin coated Falcon tissue culture inserts (pore size-1 μm; surface area 0.83 cm²) in 1 ml of growth medium. The bottom chamber of the insert assembly contained 2 ml of growth medium supplemented with the fetal human astrocyte-conditioned medium in a 1:1 (v/v) ratio (Muruganandam et al., 1997). The model was virtually impermeable for hydrophilic compounds with molecular weight>1 kDa (Muruganandam et al., 1997).

Transport studies were performed 7 days post-seeding as described previously (Muruganandam et al., 1997; Muruganandam et al., 2002). Filter inserts were rinsed with transport buffer [phosphate buffered saline (PBS) containing 5 mM glucose, 5 mM MgCl₂, 10 mM HEPES, 0.05% bovine serum albumin (BSA), pH 7.4] and allowed to equilibrate at 37° C. for 30 minutes. Experiments were initiated by adding 10 μg/ml FC5 to either apical or basolateral side of inserts containing either 0.5% gelatin-coated inserts without cells, control HCEC or HCEC pre-exposed to various pharmacological modulators for 30 min. Transport studies were conducted at 37° C. with plates positioned on a rotating platform stirring at 30-40 rpm. Aliquots (100 μl) were collected from the opposite chamber at various time intervals (5, 15, 30, 60, 90 minutes) and replaced with fresh buffer. The amount of FC5 transported across empty inserts or HCEC monolayers was determined by enzyme linked immunosorbent assay (ELISA) (see below). To control for HCEC membrane integrity and to estimate paracellular diffusion, the apical-to-basolateral and basolateral-to-apical clearance rates of [¹⁴C]-sucrose were determined and calculated essentially as described previously (Muruganandam et al., 2002; Garberg et al., 2005) across the same monolayers used for FC5 transport studies. Sample-associated radioactivity in 50 μl aliquots was measured using a Mircobeta Trilux liquid scintillation counter (Wallac, Finland).

Clearance was calculated as CI (ml)=C_{A}/C_{I}×V_{A}., where C_{I} is the initial tracer or sdAb concentration in the donor chamber, C_{A} is the tracer or sdAb concentration in the acceptor chamber, and V_{A} is the volume of the acceptor chamber. Clearance of FC5 was linear between 15 min and 60 min, while saturation was reached between 60 min and 90 min (Muruganandam et al., 2002). The effects of pharmacological agents on FC5 transmigration was subsequently assessed at 30 min. HCEC monolayer is virtually impermeable for non-selected sdAbs isolated from the same library or fluorescent dextran of similar molecular weight (Muruganandam et al., 2002).

### Laser Scanning Confocal Microscopy

A co-localization of FC5 with clathrin or caveolin-1 was studied by double immunofluorescence labeling. HCEC were first incubated with 5 μg/ml FC5 for 30 minutes, washed, fixed with 4% formaldehyde and permeabilized with 0.1% Triton X-100 for 10 minutes. Cells were then blocked with 4% goat serum for 1 hour. After blocking, cells were first incubated with anti c-Myc monoclonal antibody (1:100) for 1 hour followed by extensive washing, and then with FITC anti-mouse IgG secondary antibody (1:250) for 1 hour. After a second overnight blocking with 4% goat serum, HCEC were incubated with either anti-clathrin (1:100) or anti-caveolin-1 (1:300) polyclonal antibody for 1 hour, and then Alexa 568-conjugated anti-rabbit IgG secondary antibody (1:300) for 1 hour. Texas red-conjugated transferrin (1 μM) and cathepsin B monoclonal antibody (1:200) were used as markers for early and late endosomes, respectively. Coverslips with stained cells were washed 5 times in HBSS and mounted in fluorescent mounting medium (Dako Mississauga, Ontario).

Imaging of cells processed for double immunochemistry was performed using Zeiss LSM 410 (Carl Zeiss, Thornwood, N.Y.) inverted laser scanning microscope (LSM) equipped with an Argon\Krypton ion laser and a Plan neofluar 63X, 1.3 NA oil immersion objective. Confocal images of two fluoroprobes were obtained simultaneously to exclude artifacts from sequential acquisition, using 488 and 568 nm excitation laser lines to detect FITC (BP505-550 emission) and Texas red/Alexa 568 fluorescence (LP590 emission), respectively. All images were collected using the same laser power and pinhole size for the respective channels and processed in identical manner.

Omission of primary antibodies resulted in no staining. No cross-reactivity was observed between the primary and non-corresponding secondary antibodies.

### Cellular Fractionation

To isolate protein and lipid fractions, HCEC were washed with PBS, scraped and lyophilized. Cell remnants were dissolved in 50 mM Tris, pH 7.2. Proteins were separated from lipids with a chloroform-methanol mixture using a modified version of the Wessel and Flugge protocol (Wessel and Flugge, 1984). Before drying the lipid fraction under a stream of nitrogen gas, galactosylceramide was added as a positive control. Proteins and lipids were dissolved in 6 M urea and methanol, respectively.

Detergent-free method was used to isolate low density membrane fraction as described previously (Abulrob et al., 2004). All steps were carried out at 4° C. and all buffers were supplemented with a cocktail of protease inhibitors (Sigma). Plasma membrane fractions were prepared from five 75 cm² tissue culture flasks of confluent HCEC incubated in the presence of 5 μg/ml FC5 for 30 minutes. Each flask was washed twice with 10 ml of buffer A (0.25 M sucrose, 1 mM EDTA, and 20 mM Tricine, pH 7.8), cells were then collected by scraping in 5 ml buffer A, pelleted by centrifugation at 1400×g for 5 minutes (Beckman J-68), resuspended in 1 ml of buffer A, and homogenized by 20 up/down strokes with a Teflon glass homogenizer. Homogenized cells were centrifuged twice at 1000×g for 10 minutes (Eppendorf Centrifuge 5415C), and the two postnuclear supernatant fractions were collected, pooled, overlayed on top of 23 ml of 30% Percoll solution in buffer A and ultracentrifuged at 83,000×g for 30 minutes in a Beckman 60Ti. The pellet, representing plasma membrane fraction, was collected and sonicated 6 times at 50 J/W per second (Fisher Sonic Dismembrator 300). The sonicated plasma membrane fraction was mixed with 50% Optiprep in buffer B (0.25 M sucrose, 6 mM EDTA, and 120 mM Tricine, pH 7.8) (final Optiprep concentration, 23%). The entire solution was placed at the bottom of the Beckman SW41Ti tube, overlayed with a linear 20-10% Optiprep gradient, and centrifuged at 52,000×g for 90 minutes using SW41Ti (Beckman Instruments). The top 5 ml of the gradient was collected and mixed with 50% Optiprep in buffer B, placed on the bottom of a SW41Ti tube, overlayed with 2 ml of 5% Optiprep in buffer A and centrifuged at 52,000×g for 90 minutes. An opaque band located just above the 5% interface was designated the “caveolae fraction.” The gradient was fractionated into 1.25 ml fractions.

### SDS-Polyacrylamide Gel Electrophoresis (SDS-PAGE) and Western Immunoblot Analysis

For immunoblot detection of FC5, caveolin-1 and clathrin heavy chain proteins, each fraction of the final Optiprep gradient was resolved on SDS-polyacrylamide gels under reducing conditions. The separated proteins were electrophoretically transferred to a PVDF membrane (Immobilon P; Millipore, Nepean, Ontario). After blocking with 5% skim milk for 1 hour, the membrane was probed with HRP-conjugated anti c-Myc monoclonal antibody (dilution 1:1000), polyclonal anti-caveolin antibody (dilution 1:500) or anti-clathrin antibody (dilution 1:500) in TBS-Tween with 5% skim milk for 2 hours. ECL plus western blotting detection system was used to detect signals.

### Enzyme-Linked Immunosorbent Assay (ELISA)

To measure the amount of FC5 transmigrated across the in vitro BBB model, 50 μl aliquots collected from the appropriate compartment were immobilized overnight at room temperature in a HisGrab nickel coated 96-well plate (Pierce). After blocking the plates with 2% BSA for 2 hours at room temperature, anti-c-Myc monoclonal antibody conjugated to HRP was added at a dilution of 1:5000 for 1 hour. After washing, the bound FC5 was detected with tetramethylbenzidine (TMB)/hydrogen peroxide substrate system. The signal was measured at 450 nm on a microtiter plate reader. FC5 concentrations in collected aliquots were determined from a standard curve constructed using known FC5 concentrations.

To measure FC5 binding to HCEC protein and lipid fractions, isolated fractions were coated onto a flexible 96-well ELISA plate by drying overnight at 37° C. The ELISA plate was blocked with 0.5% BSA in PBS for 2 hours. Plates were then incubated with either FC5 antibody or with the O1 antibody against galactosylceramide (kind gift from Dr. J. Totter, University of Heidelberg, Germany). The FC5 antibody was detected with the mouse anti-myc antibody 9E10. The assay was further carried out as described.

### REFERENCES

Inclusion of a reference is neither an admission nor a suggestion that it is relevant to the patentability of anything disclosed herein.
Abulrob A., Zhang J., Tanha J., MacKenzie R. and Stanimirovic D. (2005). Single domain antibodies as blood brain barrier delivery vectors. Intern. Cong. Ser. 1277, 212-223.
Dehouck B. Fenart L., Dehouck M. P., Pierce A., Torpier G., and Cecchelli R. (1997) A new function for the LDL receptor: transcytosis of LDL across the blood-brain barrier. J. Cell Biol. 138, 877-889.
Demeule M., Poirier J. Jodoin J., et al. (2002) High transcytosis of melanotransferrin (P97) across the blood-brain barrier. J. Neurochem. 83, 924-933.
Drin G., Cottin S., Blanc E., Rees A. R. and Temsamani J. (2003). Studies on the internalization mechanism of cationic cell-penetrating peptides. J. Biol. Chem. 278, 31192-31201.
Drin G., Rousselle C., Scherrmann J. M., Rees A. R. and Temsamani J. (2002). Peptide delivery to the brain via adsorptive-mediated endocytosis: advances with SynB vectors. AAPS PharmSci. 4, E26.
Fillebeen C., Descamps L., Dehouck M. P., Fenart L., Benaissa M., Spik G., Cecchelli R., and Pierce A. (1999) Receptor-mediated transcytosis of lactoferrin through the blood-brain barrier. J. Biol. Chem. 274, 7011-7.
Hayes G. R., Enns C. A. and Lucas J. J. (1992). Identification of the O-linked glycosylation site of the human transferrin receptor. Glycobiology. 2, 355-359.
Moos T. and Morgan E. H. (2001). Restricted transport on anti-transferrin receptor antibody (OX26) through the blood-brain barrier in the rat. J. Neurochem. 79, 119-129.
Muruganandam A., Tanha J., Narang S, and Stanimirovic D. (2002). Selection of phage-displayed llama single-domain antibodies that transmigrate across human blood-brain barrier endothelium. FASEB J. 16, 240-242.
Qian Z. M., Li H., Sun H. and Ho K. (2002). Targeted drug delivery via the transferrin receptor-mediated endocytosis pathway. Pharmacol. Rev. 54, 561-587.
Suzuki T., Futaki S., Niwa M., Tanaka S., Ueda K. and Sugiura Y. (2002). Possible existence of common internalization mechanisms among arginine-rich peptides. J. Biol. Chem. 277, 2437-2443.
Tanha J., Muruganadam A. and Stanimirovic D. (2003). Phage display technology for identifying specific antigens on brain endothelial cells. Methods Mol. Med. 89, 435-449.
Wessel D. and Flugge U. I. (1984). A method for the quantitative recovery of protein in dilute solution in the presence of detergents and lipids. Anal. Biochem. 138, 141-143.
Zhang J., Tanha J., Hirama T., Khieu N. H., To R., Tong-Sevinc H., Stone E., Brisson J. R. and MacKenzie C. R. (2004). Pentamerization of single-domain antibodies from phage libraries: a novel strategy for the rapid generation of high-avidity antibody reagents. J. Mol. Biol. 335, 49-56.
Zhang J., Li Q., Nguyen T., Tremblay T., Stone E., Kelly J. and MacKenzie R. (2004). A pentavalent single domain antibody approach to tumor antibody discovery and the development of novel proteomics reagents. J. Mol. Biol. 341, 161-9.

1-15. (canceled)

16. A method of identifying a candidate agent capable of transmigration across the blood-brain barrier comprising:
incubating an agent of interest, with the proviso that said agent is not a single domain antibody (sdAb), with a peptide comprising an amino acid sequence having at least 75% identity to an amino acid sequence selected from the group consisting of amino acids 67-323 as set forth in SEQ ID NO: 3; amino acids 67-287 as set forth in SEQ ID NO: 4; and amino acids 1-204 as set forth in SEQ ID NO: 5;
detecting binding between said agent and said peptide; and
determining whether there is an increase in internalization of the agent, thereby identifying the candidate agent.

17. The method of claim 16, wherein the agent of interest is an antibody.

18. The method of claim 16, wherein the agent of interest is a small molecule.

19. The method of claim 16, wherein the peptide is a glycosylated peptide.

20. The method of claim 16, wherein the peptide comprises an amino acid sequence having at least 75% identity to amino acids 1-323 of SEQ ID NO. 3.

21. The method of claim 16, wherein the peptide comprises an amino acid sequence having at least 75% identity to amino acids 67-323 of SEQ ID NO. 3.

22. The method of claim 16, wherein the peptide comprises an amino acid sequence having at least 75% identity to amino acids 1-361 of SEQ ID NO. 3.

23. A method of identifying an agent capable of interacting with transmembrane domain protein 30A (TMEM30A) comprising:
incubating an agent of interest, with the proviso that said agent is not a single domain antibody (sdAb), with a peptide comprising an amino acid sequence having at least 75% identity to an amino acid sequence selected from the group consisting of amino acids 67-323 as set forth in SEQ ID NO: 3; amino acids 67-287 as set forth in SEQ ID NO: 4; and amino acids 1-204 as set forth in SEQ ID NO: 5; and
detecting binding between said agent and said peptide.

24. The method of claim 23, wherein the agent of interest is an antibody.

25. The method of claim 23, wherein the agent of interest is a small molecule.

26. The method of claim 23, wherein the peptide is a glycosylated peptide.

27. The method of claim 23, wherein the peptide comprises an amino acid sequence having at least 75% identity to amino acids 1-323 of SEQ ID NO. 3.

28. The method of claim 23, wherein the peptide comprises an amino acid sequence having at least 75% identity to amino acids 67-323 of SEQ ID NO. 3.

29. The method of claim 23, wherein the peptide comprises an amino acid sequence having at least 75% identity to amino acids 1-361 of SEQ ID NO. 3.

30. An in vitro method of identifying an agent capable of interacting with transmembrane domain protein 30A (TMEM30A) comprising:
incubating an agent of interest with a peptide comprising an amino acid sequence having at least 75% identity to an amino acid sequence selected from the group consisting of amino acids 67-323 as set forth in SEQ ID NO: 3; amino acids 67-287 as set forth in SEQ ID NO: 4; and amino acids 1-204 as set forth in SEQ ID NO: 5, wherein the incubating occurs in a substantially cell free system; and
detecting binding between said agent and said peptide.

31. The method of claim 30, wherein the agent of interest is an antibody.

32. The method of claim 30, wherein the agent of interest is a single domain antibody.

33. The method of claim 30, wherein the agent of interest is a small molecule.

34. The method of claim 30, wherein the peptide is a glycosylated peptide.

35. The method of claim 30, wherein the peptide comprises an amino acid sequence having at least 75% identity to amino acids 1-323 of SEQ ID NO. 3.

36. The method of claim 30, wherein the peptide comprises an amino acid sequence having at least 75% identity to amino acids 67-323 of SEQ ID NO. 3.

37. The method of claim 30, wherein the peptide comprises an amino acid sequence having at least 75% identity to amino acids 1-361 of SEQ ID NO. 3.

38. The method of claim 30, wherein the agent of interest is not a single domain antibody (sdAb).

39. A method of identifying an agent capable of transmigration across the blood-brain barrier comprising:
providing a cell that is transformed with a nucleic acid and expresses a peptide comprising at least 75% identity to an amino acid sequence selected from the group consisting of amino acids 67-323 as set forth in SEQ ID NO: 3; amino acids 67-287 as set forth in SEQ ID NO: 4; and amino acids 1-204 as set forth in SEQ ID NO: 5;
incubating an agent of interest with the cell that expresses the peptide;
detecting binding between the agent and the peptide; and
determining whether there is an increase in internalization of the agent, thereby identifying the candidate agent.

40. The method of claim 39, wherein the agent of interest is an antibody.

41. The method of claim 39, wherein the agent of interest is a single domain antibody.

42. The method of claim 39, wherein the agent of interest is a small molecule.

43. The method of claim 39, wherein the peptide is a glycosylated peptide.

44. The method of claim 39, wherein the peptide comprises an amino acid sequence having at least 75% identity to amino acids 1-323 of SEQ ID NO. 3.

45. The method of claim 39, wherein the peptide comprises an amino acid sequence having at least 75% identity to amino acids 67-323 of SEQ ID NO. 3.

46. The method of claim 39, wherein the peptide comprises an amino acid sequence having at least 75% identity to amino acids 1-361 of SEQ ID NO. 3.

47. The method of claim 39, wherein the agent of interest is not a single domain antibody (sdAb).

48. A method of identifying an agent capable of interacting with transmembrane domain protein 30A (TMEM30A) comprising:
providing a cell that is transformed with a nucleic acid and expresses a peptide comprising at least 75% identity to an amino acid sequence selected from the group consisting of amino acids 67-323 as set forth in SEQ ID NO: 3; amino acids 67-287 as set forth in SEQ ID NO: 4; and amino acids 1-204 as set forth in SEQ ID NO: 5;
incubating an agent of interest with the cell that expresses the peptide; and
detecting binding between said agent and said peptide.

49. The method of claim 48, wherein the agent of interest is an antibody.

50. The method of claim 48, wherein the agent of interest is a single domain antibody.

51. The method of claim 48, wherein the agent of interest is a small molecule.

52. The method of claim 48, wherein the peptide is a glycosylated peptide.

53. The method of claim 48, wherein the peptide comprises an amino acid sequence having at least 75% identity to amino acids 1-323 of SEQ ID NO. 3.

54. The method of claim 48, wherein the peptide comprises an amino acid sequence having at least 75% identity to amino acids 67-323 of SEQ ID NO. 3.

55. The method of claim 48, wherein the peptide comprises an amino acid sequence having at least 75% identity to amino acids 1-361 of SEQ ID NO. 3.

56. The method of claim 48, wherein the agent of interest is not a single domain antibody (sdAb).


SEQUENCE LISTING (28 sequences)

SEQ ID NO: 1 - DNA, 384 bp, human
gaattttatggagaaagggattacaagatgtatgagtataatgacttgctaacctttcaggattcagagaaagatgaagaaagaccatatctaaataatacacttcatcattttcatgtgtataaatgcttaaagtaccatctttgttgaggtggttcatgtatccagtttatccagtacagttatttgtcaagcttagctttgatttcaaaggacacgcttaccttgtctggcataagaattaatgctcatgtctgcagtggttgggtaggtcctgcttaggagaattaaaaaattcctctttccgtttggttgaatgttgcagtcaggaaccccaactcacttggaatgttttcatatgtaatcatttcccttgaagcttat

SEQ ID NO: 2 - DNA, 1090 bp, human
  misc_feature (141)..(1226): coding sequence
atggcgatgaactataacgcgaaggatgaagtggacggtgggcccccgtgtgctccggggggcaccgcgaagactcggagaccggataacacggccttcaaacagcaacggctgccagcttggcagcccatccttacggctggcacggtgctacctattttcttcatcatcggtctcatcttcattcccatcggcattggcatttttgtcacctccaacaacatccgcgagatcgagattgattataccggaacagagccttccagtccctgtaataaatgtttatctccggatgtgacaccttgcttttgtaccattaacttcacactggaaaagtcatttgagggcaacgtgtttatgtattatggactgtctaatttctatcaaaaccatcgtcgttacgtgaaatctcgagatgatagtcaactaaatggagattctagtgctttgcttaatcccagtaaggaatgtgaaccttatcgaagaaatgaagacaaaccaattgctccttgtggagctattgccaacagcatgtttaatgatacattagaattgtttctcattggcaatgattcttatcctatacctatcgctttgaaaaagaaaggtattgcttggtggacagataaaaatgtgaaattcagaaatccccctggaggagacaacctggaagaacgatttaaaggtacaacaaagcctgtgaactggcttaaaccagtttacatgctggattctgacccagataataatggattcataaatgaggattttattgtttggatgcgtactgcagcattacctacttttcgcaagttgtatcgtcttatagaaaggaaaagtgatttacatccaacattaccagctggccgatactctttgaatgtcacatacaattaccctgtacattattttgatggacgaaaacggatgatcttgagcactatttcatggatgggaggaaaaaatccatttttggggattgcttacatcgctgttggatccatctccttccttctgggagttgtactgctagtaattaatcataaatatagaaacagtagtaatacagctgacattaccatttaatttt

SEQ ID NO: 3 - protein, 361 aa, human
  CARBOHYD (180)..(180): glycosylation site
MAMNYNAKDEVDGGPPCAPGGTAKTRRPDNTAFKQQRLPAWQPILTAGTVLPIFFIIGLIFIPIGIGIFVTSNNIREIEIDYTGTEPSSPCNKCLSPDVTPCFCTINFTLEKSFEGNVFMYYGLSNFYQNHRRYVKSRDDSQLNGDSSALLNPSKECEPYRRNEDKPIAPCGAIANSMFNDTLELFLIGNDSYPIPIALKKKGIAWWTDKNVKFRNPPGGDNLEERFKGTTKPVNWLKPVYMLDSDPDNNGFINEDFIVWMRTAALPTFRKLYRLIERKSDLHPTLPAGRYSLNVTYNYPVHYFDGRKRMILSTISWMGGKNPFLGIAYIAVGSISFLLGVVLLVINHKYRNSSNTADITI

SEQ ID NO: 4 - protein, 325 aa, human
MAMNYNAKDEVDGGPPCAPGGTAKTRRPDNTAFKQQRLPAWQPILTAGTVLPIFFIIGLIFIPIGIGIFVTSNNIREIEGNVFMYYGLSNFYQNHRRYVKSRDDSQLNGDSSALLNPSKECEPYRRNEDKPIAPCGAIANSMFNDTLELFLIGNDSYPIPIALKKKGIAWWTDKNVKFRNPPGGDNLEERFKGTTKPVNWLKPVYMLDSDPDNNGFINEDFIVWMRTAALPTFRKLYRLIERKSDLHPTLPAGRYSLNVTYNYPVHYFDGRKRMILSTISWMGGKNPFLGIAYIAVGSISFLLGVVLLVINHKYRNSSNTADITI

SEQ ID NO: 5 - protein, 242 aa, human
MYYGLSNFYQNHRRYVKSRDDSQLNGDSSALLNPSKECEPYRRNEDKPIAPCGAIANSMFNDTLELFLIGNDSYPIPIALKKKGIAWWTDKNVKFRNPPGGDNLEERFKGTTKPVNWLKPVYMLDSDPDNNGFINEDFIVWMRTAALPTFRKLYRLIERKSDLHPTLPAGRYSLNVTYNYPVHYFDGRKRMILSTISWMGGKNPFLGIAYIAVGSISFLLGVVLLVINHKYRNSSNTADITI

SEQ ID NO: 6 - protein, 257 aa, human
GIFVTSNNIREIEIDYTGTEPSSPCNKCLSPDVTPCFCTINFTLEKSFEGNVFMYYGLSNFYQNHRRYVKSRDDSQLNGDSSALLNPSKECEPYRRNEDKPIAPCGAIANSMFNDTLELFLIGNDSYPIPIALKKKGIAWWTDKNVKFRNPPGGDNLEERFKGTTKPVNWLKPVYMLDSDPDNNGFINEDFIVWMRTAALPTFRKLYRLIERKSDLHPTLPAGRYSLNVTYNYPVHYFDGRKRMILSTISWMGGKNP

SEQ ID NO: 7 - protein, 40 aa, human
RRNEDKPIAPCGAIANSMFNDTLELFLIGNDSYPIPIALK

SEQ ID NO: 8 - protein, 140 aa, human
RRNEDKPIAPCGAIANSMFNDTLELFLIGNDSYPIPIALKKKGIAWWTDKNVKFRNPPGGDNLEERFKGTTKPVNWLKPVYMLDSDPDNNGFINEDFIVWMRTAALPTFRKLYRLIERKSDLHPTLPAGRYSLNVTYNYP

SEQ ID NO: 9 - protein, 18 aa, human
NFYQNHRRYVKSRDDSQL

SEQ ID NO: 10 - protein, 11 aa, human
APCGAIANSMF

SEQ ID NO: 11 - protein, 11 aa, human
APCGAIANSLF

SEQ ID NO: 12 - protein, 13 aa, human
DFIVWMRTAALPT

SEQ ID NO: 13 - protein, 13 aa, human
DFVVWMRTAALPT

SEQ ID NO: 14 - protein, 16 aa, human
MGGKNPFLGIAYIAVG

SEQ ID NO: 15 - protein, 16 aa, human
MGGKNPFLGIAYLVVG

SEQ ID NO: 16 - DNA, 24 bp, artificial
  PCR foward primer for TMEM30A
gaagactcggagaccggataacac

SEQ ID NO: 17 - DNA, 25 bp, artificial
  TMEM30A expression reverse primer
cagtacaactcccagaaggaaggag

SEQ ID NO: 18 - protein, 6 aa, human
SSPCNK

SEQ ID NO: 19 - protein, 4 aa, human
LIER

SEQ ID NO: 20 - protein, 8 aa, human
HSFDGRKR

SEQ ID NO: 21 - protein, 10 aa, human
NYPVHSFDGR

SEQ ID NO: 22 - DNA, 31 bp, artificial
  his cloning forward primer
tctcgaattcatggcgatgaactataacgcg

SEQ ID NO: 23 - DNA, 31 bp, artificial
  his cloning reverse primer
tctcaccggtaatggtaatgtcagctgtatt

SEQ ID NO: 24 - DNA, 18 bp, artificial
  TMEM30A SP1 primer
tctcgatctcgcggatgc

SEQ ID NO: 25 - DNA, 18 bp, artificial
  TMEM30A SP2 primer
catccaacattaccagct

SEQ ID NO: 26 - DNA, 18 bp, artificial
  TMEM30A SP3 primer
cggatgatcttgagcact

SEQ ID NO: 27 - DNA, 30 bp, artificial
  sequencing primer
gtgaaaaaattattattattcgcaattcct

SEQ ID NO: 28 - DNA, 20 bp, artifical
ccctcatagttagcgtaacg